(19) United States
(12) Patent Application Publication
(10) Pub. No.: US 2026/0218122 A1
(43) Pub. Date: Jul. 30, 2026

(54) SYSTEMS AND METHODS OF PREPARING AND DELIVERING OOCYST SOLUTIONS

(71) Applicant: TARGAN, Inc., Raleigh, NC (US)

(72) Inventors: James Earl HUTCHINS, Durham, NC (US); Ramin KARIMPOUR, Raleigh, NC (US); Elizabeth TURPIN, Raleigh, NC (US); Stephen WOLFE, Raleigh, NC (US); Joshua Steven GOFF, Raleigh, NC (US); Ryan D. BOEHM, Durham, NC (US)

(73) Assignee: TARGAN, Inc., Raleigh, NC (US)

(21) Appl. No.: 19/578,410

(22) Filed: Mar. 25, 2026

Related U.S. Application Data
(63) Continuation-in-part of application No. 17/258,867, filed on Jul. 10, 2019, now Pat. No. 12,605,482.
(60) Provisional application No. 62/696,261, filed on Jul. 10, 2018.

(51) Int. Cl. C12N 5/00; C12M 1/26; C12N 5/07

(52) U.S. Cl. CPC C12N 5/0006; C12M 33/08; C12N 5/0601

(57) ABSTRACT

The present disclosure provides for disrupting the outer membrane of an oocyst in solution and delivering the solution to an animal. The system includes a vessel containing unbroken oocysts in solution, an oocyst processing chamber, and a delivery outlet. The unbroken oocysts are moved from the vessel through the processing chamber and a portion of the oocyst membranes are disrupted releasing sporocysts, the resulting solution is moved from the processing chamber into the delivery outlet where the solution is delivered to an animal. Methods of vaccination (e.g. against Eimeria infection) are also provided. Another application relates to cell lysis processes associated with deriving subcellular pharmaceutical/biopharmaceutical substances from various cell types in a drug manufacturing environment. This may also be applied to: disruption of cells in the production of subcellular ingredients for human nutritional supplements/food, animal feed supplements or feed; reclamation of phosphorous and nitrogen from cells in waste streams.

### CROSS REFERENCE TO RELATED APPLICATIONS

This application claims the benefit of U.S. Provisional Application 62/696,261 filed Jul. 10, 2018, Hutchins et al., which is hereby incorporated by reference in its entirety. The present application is a continuation-in-part of co-pending U.S. patent application Ser. No. 17/258,867 filed on Jan. 8, 2021 by inventors James Hutchins et. al., which entered the national stage under 35 U.S.C. 371 from International Application No. PCT/US19/41178 filed on Jul. 10, 2019 by inventors James Hutchins et al. The foregoing applications are incorporated herein by reference in their entirety.

### FIELD

The present disclosure provides systems and methods for disrupting the outer membrane of an oocyst in solution and delivering the solution to an animal. The system includes a vessel containing unbroken oocysts in solution, an oocyst processing chamber, and a delivery outlet. The unbroken oocysts are moved from the vessel through the processing chamber and a portion of the oocyst membranes are disrupted releasing sporocysts, and the resulting solution is moved from the processing chamber into the delivery outlet where the solution is delivered to an animal.

### BACKGROUND

### a. Introduction

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Vaccines are an important component of protecting humans and animals from pathogenic microorganisms, including viruses, bacteria, and parasites. Briefly, a vaccine stimulates the immune system to recognize a specific pathogen, thereby making a defense system that protects against future encounters with that microorganism in nature. Vaccines may be divided into several major classes: inactivated or killed vaccines; subunit, recombinant, polysaccharide, and conjugate vaccines; messenger RNA vaccines; toxoid vaccines; viral-vectored vaccines; wildtype vaccines and attenuated or modified-live vaccines. The wildtype and attenuated vaccines give the recipient animal a mild infection. The mild infection often produces an immune response so as to prevent a greater, perhaps lethal infection from occurring in the future.

### b. Apicomplexa, Eimeria, Coccidiosis, and Vaccines

Apicomplexa is a phylum of unicellular and spore forming parasites with a complex life cycle. Well-known human diseases caused by Apicomplexa include babesiosis (Babesia), cryptosporidiosis (Cryptosporidium parvum), malaria (Plasmodium), and toxoplasmosis (Toxoplasma gondii). Apicomplexan diseases also affect animals and livestock. Some Apicomplexa, such as Cryptosporidium parvum and Toxoplasma gondii, affect both humans and animals. Other Apicomplexa such as Eimeria or Theileria only affect animals. The Apicomplexa life cycle is complicated in that it has both sexual and asexual reproductive stages. The life cycle often consists of both a stage where it is excreted into the environment, and other stages that occur within the animal host. For many Apicomplexa some stages of the life cycle take place in one host species and other stages take place in another host species. On the other hand, the Apicomplexan parasite, Eimeria, is generally host specific and is monoxenous, in that its the life cycle is specific for a single host species.

Eimeria causes coccidiosis in the wild and domesticated vertebrates such as cattle, chickens, fish, goats, pigs, rabbits, reptiles, sheep, and turkey. Different Eimeria species have a preferred section of the gastrointestinal (GI) tract where they reproduce and cause damage to the epithelium of the GI tract.

Portions of the life cycle of an Eimeria oocyst whether wildtype or attenuated are illustrated in FIG. 1A & FIG. 1B. FIG. 1A portrays an overview of the external process that occurs with Eimeria oocyst uptake in chickens. The day-of-hatch chicken is first inoculated with vaccine that contains at least one sporulated oocyst per dose (A). The sporulated oocyst(s) is then processed within the digestive tract of the chicken as shown in greater detail in FIG. 1B. The infection continues through multiple life stages, eventually resulting in the formation of unsporulated oocysts that are excreted in the chicken's feces (B). Following excretion from the bird, the unsporulated oocysts are then exposed to heat, moisture and oxygen in the environment, and become sporulated over the course of several days (C). The oocysts are not infective until they are sporulated. These sporulated oocysts are then ingested by the chicken, and the cycle repeats.

FIG. 1B portrays an enlarged view of the internal processes that occur with Eimeria oocyst uptake in chickens. The boxed region shows a simplified depiction of the oocyst reproductive life cycle, wherein the sporulated oocyst, containing four sporocysts, is cracked, releasing sporocysts (D). Within each sporocyst are two sporozoites. Enzymatic reactions within the bird's intestines digest the endcap of the sporocyst wall (not shown), releasing the sporozoites. The motile sporozoites then seek out and infect intestinal cells (E) in different regions of the intestines in a species-specific manner. For example, in chickens E. acervulina infects the upper intestine, E. maxima infects the small intestine, and E. tenella infects the caecum.

Following infection of intestinal cells by sporozoites, the life cycle of the parasite continues through several stages of asexual reproduction. These cycles consist of several rounds of reproduction and amplification that result in a massive increase in Eimeria presence within their select regions of the intestinal tract. After amplification brought on by the asexual reproduction stages, sexual reproduction occurs and results in the production of oocysts, that will then be shed in the feces of a chicken and consumed by another chicken as depicted in FIG. 1A.

The complete process takes approximately 7 days, with exact lengths of time varying by species. The excystation process and subsequent invasion of a host cell occur approximately between day 0 and day 3. The asexual reproduction cycle occurs approximately between day 3 and day 5. The sexual reproduction phase and subsequent shedding of the oocyst in the feces occur approximately between day 5 and day 7.

Coccidiosis is a common disease in poultry. Control of coccidiosis has typically been achieved using ionophores or chemicals in the feed. Alternative control measures are currently being sought for a variety of reasons including, but not limited to, the high cost of ionophores and chemicals, the environmental impact of ionophores and other chemicals, consumer demand for pesticide- and antibiotic-free poultry, and the development of resistant coccidia. Vaccines for coccidiosis have the potential to drastically reduce or eliminate the need for ionophores or chemicals in feed for coccidiosis control. Vaccines are not widely used due to the lack of uniformity with mass vaccine application. As presently delivered, Eimeria vaccines in poultry result in inefficient first round infectivity and immunity and typically result in a large naïve population susceptible to disease. The subsequent naïve population depends on recycling in the grow out farms to induce immunity. Oocysts output from birds infected in the first-round yield massive infection of the naïve population. Resolution of naivety yields high oocyst output in the period following the first-round infection, which results in susceptibility to secondary bacterial infections, such as necrotic enteritis, requiring antibiotics for resolution. Effective vaccination of all birds at the day of hatch would avoid the morbidity, mortality and lack of weight gain associated with Eimeria infection. See PCT Publication WO 2017/083663A1, Karimpour.

Currently the global impact of coccidiosis due to poor performance, morbidity and mortality is estimated at $300 million. In addition, an estimated $90 million is spent in the US and $3 billion globally for coccidiosis control annually (5m Editor, 2013, High Cost of Coccidiosis in Broilers, The Poultry Site).

The process of cracking open the oocyst membrane is presently thought to be facilitated by grinding of oocysts in contact with grit and feed in the gizzard of the chicken. Delivery of oocysts to unvaccinated or naïve birds at a hatchery results in inefficient vaccination, as the gut does not contain sufficient feed or grit to assist the process of cracking the oocyst wall to release sporocysts. As such, it would be preferable to deliver sporocysts directly to the day-old hatchlings as they may not have any grit or food in their digestive system to help break down the oocyst wall and release the sporocysts. Direct delivery of sporocysts to naïve birds at a hatchery can improve efficiency of vaccination, as the vaccine can be infective even in the absence of grit or food. Young chickens do have the capability of processing released sporocysts to the infected sporozoite stage in the intestines. This sporocyst vaccine strategy may prevent the need for recycling and secondary rounds of infection for the development of full immunity.

Some attempts have previously been made to crack the oocyst wall for purposes of generating a vaccine solution that is manufactured and shipped as a sporocyst vaccine. For example, others have disclosed grinding or shaking oocysts with glass beads. Additionally, European Patent 2,111, 243 B1 (Hutchins et al., Embrex, Inc.) discloses methods to release sporocysts from oocysts using microchannels.

However, prior attempts to disrupt the oocyst membrane have been done in the context of preparing a solution for cryopreservation, freezing, storing, subsequent delivery, and thawing. Specifically, prior methods for producing sporocyst based vaccines have the disadvantage that the resulting sporocyst must be suspended in a cryopreservative solution and stored in liquid nitrogen long term. Such processes are well known in the art of parasitology for the preservation of master seed lines in the sporocyst form. It is also appreciated that recovery of viable sporocysts after cryopreservation is low, often only 5-10%. The real time generation and delivery of sporocysts avoids the need for cryopreservation and the resulting low recovery of viable organisms.

Another challenge is to disrupt the oocyst membrane without damaging the sporocysts. It becomes even more difficult when working with vaccines that are composed of multiple Eimeria species that are different sizes and have varying thicknesses and durabilities of their protective walls. Thus, the conditions needed to break the walls for a small rupture-resistant species such as E. acervulina may either damage previously released sporocysts or be too rigorous for a large more rupture-susceptible species like E. maxima.

There have been no attempts to crack the oocyst wall at the time of delivery or in an in-line process where the newly released sporocysts are delivered directly and immediately to the intended recipient.

### SUMMARY OF THE DISCLOSURE

The embodiments herein are directed to systems and methods pertaining to the in situ release of sporocysts for improved vaccination. Some embodiments described herein are directed to systems and methods for disrupting the membrane of an oocyst and delivering the membrane and its contents, viable sporocysts, to an animal.

Other embodiments described herein are directed to an oocyst solution delivered in two parts to create a gel mixture. See U.S. patent application Ser. No. 17/258,867 filed on Jan. 8, 2021 by inventors James Hutchins et. al., which is incorporated herein in its entirety. One embodiment is directed to a method of vaccinating an animal against Eimeria. The method includes the steps of providing an oocyst-based solution, causing viable sporocysts to be released from oocysts, and delivering the solution containing released sporocysts to the animal.

Another embodiment is directed to a system for rupturing the outer membrane of an oocyst and subsequently delivering it to an animal in real-time. The system includes a vessel containing unbroken oocysts in solution, an oocyst processing chamber, and a delivery outlet. The unbroken oocysts are moved from the vessel through the processing chamber and a portion of the oocysts' membranes are ruptured releasing sporocysts. The resulting solution is moved from the processing chamber into the delivery outlet where the solution is delivered to an animal.

A further embodiment is directed to a method of rupturing oocysts at the time of delivery to an animal. The method includes the steps of providing a first vessel for containing a volume of unbroken oocysts in solution, a processing chamber, and a delivery device. The method further includes moving the solution of unbroken oocysts from the first vessel into the processing chamber and passing the solution through the processing chamber, whereby at least a portion of sporocysts are released into the solution. The method also includes moving the solution from the processing chamber to the delivery device where the processed solution containing released sporocysts is delivered to an animal.

Still another embodiment is directed to a system for delivering an oocyst solution to an animal.

A preferred commercial bird to be vaccinated by the method of the invention is a chicken.

A preferred composition to be administered to a chicken comprises sporocysts, or a mixture of sporocysts and oocysts, of one or more species of Eimeria selected from the group consisting of E. tenella, E. acervulina, E. maxima, E. necatrix, E. mitis, E. praecox, E. hagani, E. mivati, and E. brunetti.

Another preferred commercialized bird to be vaccinated by the method of the invention is a turkey. A preferred composition to be administered to a turkey comprises sporocysts, or a mixture of sporocysts and oocysts, of one or more species of Eimeria selected from the group consisting of E. meleagrimitis, E. adenoeides, E. gallopavonis, E. dispersa, E. meleagridis, E. innocua, and E. subrotunda.

### BRIEF DESCRIPTION OF THE FIGURES

Having thus described various embodiments of the present disclosure in general terms, reference will now be made to the accompanying drawings, which are not drawn to scale and do not include all components of the system.

FIG. 1A is a graphic representation of the life cycle of Eimeria oocysts and vaccines.

FIG. 1B is a graphic representation of the life cycle of Eimeria oocysts in a chicken.

FIG. 2 is schematic drawing of the first embodiment (high-pressure homogenizer).

FIG. 3 is an enlarged view of the processing system of the first embodiment.

FIG. 4 is a schematic drawing of the second embodiment (bead treatment).

FIG. 5 is a schematic drawing of the third embodiment (sonication).

FIG. 6 is a schematic drawing of the fourth embodiment (rotor-stator mixer).

FIG. 7 is a schematic drawing of the fifth embodiment (vibrating plates).

FIG. 8 is a schematic drawing for the sixth embodiment (hydrodynamic cavitation).

FIG. 9 is a schematic drawing of the seventh embodiment (high-pressure spray).

FIG. 10A is an isometric view with hidden lines of an orifice plate with multiple orifices.

FIG. 10B is a front view of an orifice plate with multiple orifices.

FIG. 10C is a front view of an orifice plate with multiple orifices expelling liquid.

FIG. 10D is a section view of the orifice plate shown in FIG. 10C while it is expelling liquid.

FIG. 10E is an isometric view with hidden lines of a single conical nozzle.

FIG. 10F is a front view of a single conical nozzle.

FIG. 10G is a front view of a single conical nozzle expelling liquid.

FIG. 10H is a section view of the conical nozzle shown in FIG. 10G while it is expelling liquid.

FIG. 11 is a schematic drawing of the ninth embodiment (vibrating rods).

FIG. 12A is a cross-sectional view of the processing system of one embodiment.

FIG. 12B is a cross-sectional view of the processing system of another embodiment.

FIG. 12C is a cross-sectional view of the processing system of another embodiment.

FIG. 12D is a cross-sectional view of the processing system of another embodiment.

FIG. 13A is an isometric view of the processing system of one embodiment.

FIG. 13B is an isometric view of the processing system of another embodiment.

FIG. 13C is an isometric view of the processing system of another embodiment.

FIG. 13D is an isometric view of the processing system of another embodiment.

FIG. 14 is a schematic drawing of the tenth embodiment (a single vibrating rod).

FIG. 15 is a schematic drawing of the tenth embodiment (a split vibrating rod).

FIG. 16 is a schematic drawing of the tenth embodiment (concentric cylinders with a central rod).

FIG. 17 is a view of a flow chart of a method of optimization of flow rate for both percent disruption and recovery of viable sporocysts as evidenced by oocyst output of treated birds according to at least one embodiment.

### DETAILED DESCRIPTION OF THE DISCLOSURE

Various aspects of the present disclosure will be described more fully hereinafter with reference to the accompanying drawings, in which some, but not all aspects of the disclosure are shown. Indeed, this disclosure may be embodied in many different forms and should not be construed as limited to the aspects set forth herein, rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. All publications, patent applications, patents, and other references mentioned herein are incorporated herein by reference in their entireties.

### a. Definitions

The terminology used herein is for the purpose of describing particular embodiments only and is not intended to be limiting of the invention.

As used herein the term Eimeria means and includes Eimeria species infecting chickens consisting of E. maxima, E. mitis, E. tenella, E. acervulina, E. brunetti, E. necatrix, E. praecox, E. hagani, E. mivati, and any combination thereof. Eimeria includes species infecting turkeys such as E. meleagrimitis, E. adenoeides, E. gallopavonis, E. dispersa, E. innocua, E. meleagridis, and E. subrotunda, and any combination thereof. Eimeria also includes species infecting cattle such as E. zuernii, E. bovis, E. ellipsoidalis, and any combination thereof. Eimeria also include E. ahsata, E. bakuensis, E. crandallis, E. faurei, E. granulosa, E. intricata, E. marsica, E. ovinoidalis, E. pallida, E. parva, E. weybridgensis, and any combination thereof. Furthermore, the term Eimeria includes E. intestinalis, E. vejdovskyi, E. piriformis, E. coecicola, E. irresidua, E. flavescens, E. exigua, E. magna, E. perforans, E. media, E. stiedae, and any combination thereof.

The terms “animal” and “animal subjects” include but are not limited to mammalian and/or avian subjects. Suitable mammalian subjects include but are not limited to primate subjects (e.g., human subjects and non-human primate subjects such as simian), porcine, bovine (e.g., cattle), caprine, equine, feline, ovine, canine, murine (e.g., mouse, rat) and lagomorph subjects.

The terms “avian” and “avian subjects” (i.e., “bird” and “bird subjects”), as used herein, are intended to include males and females of any avian species, but are primarily intended to encompass poultry that are commercially raised for eggs, meat or as pets. Accordingly, the terms “avian” and “avian subject” are particularly intended to encompass but not be limited to chickens, turkeys, ducks, geese, quail, pheasant, parakeets, parrots, cockatoo, cockatiel, ostrich, emu and the like. In particular embodiments, the avian subject is a chicken or a turkey.

The real-time delivery of the solutions described herein means a system or method in which oocyst membranes are disrupted or otherwise broken so that the content therein is no longer contained within the oocyst membrane. The systems and methods herein are understood to be delivered within about a 24-hour period at room temperature, or within a 5-day period with refrigeration. In a hatchery, vaccine is typically introduced, stored, and delivered through a system over the course of a 4 to 8-hour shift.

As used herein, the terminology “percent reduction of oocysts” is defined as the disrupting of oocyst membranes to release internal components including sporocysts. For example, a 90% reduction in oocysts results in 10% of residual oocysts and a 90% conversion of oocysts to released sporocysts.

As used herein, the term “preening” or “preen” is defined as the act of a chicken, or other animal, ingesting oocysts, or other materials, through the act of grooming oneself, or another animal, and subsequently consuming the preened material to initiate infection.

As used herein, the term “take”, “percent take”, or “% take”, within the context of vaccine infectivity, is defined as the subject having been shown to be positive for an Apicomplexan infection, including but not limited to, Eimeria following vaccination.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. The sequence of operations (or steps) is not limited to the order presented in the claims or figures unless specifically indicated otherwise.

Throughout the present specification, the terms “about” and/or “approximately” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than #1%, or any other value or range of values therein or there below. Alternatively, depending on the context, the term “about” may mean±one half a standard deviation, ±one standard deviation, or ±two standard deviations. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

### a. Specific Embodiments

### i. First Embodiment—High-Pressure Homogenization

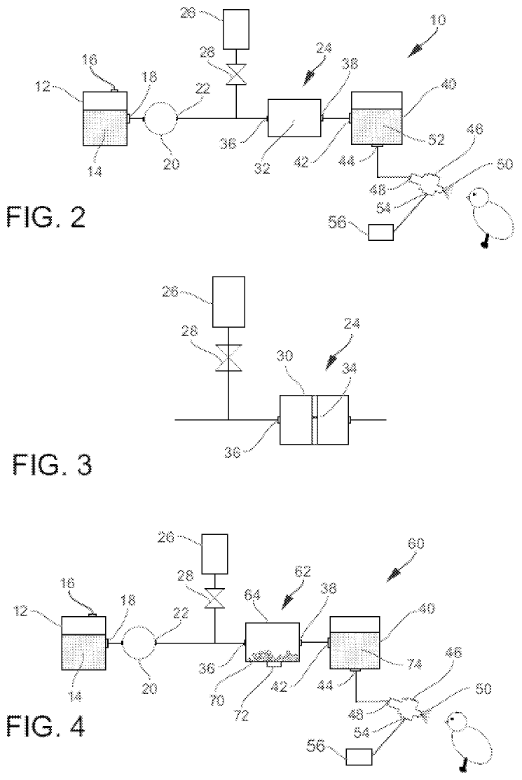

The first embodiment 10 is shown in FIG. 2. The first embodiment 10 includes a first reservoir 12. The first reservoir 12 is designed to hold a volume of solution 14. The solution 14 includes a vaccine (not shown) suspended in the solution. The solution 14 of the first embodiment 10 contains the Eimeria species oocyst-based vaccine for delivery to poultry, namely day-old hatchlings. Optionally, the solution 14 includes proteolytic enzymes. The proteolytic enzymes enable more effective uptake of the vaccine in the digestive tract of the hatchling, which will be explained in more detail below.

With regard to the solution 14, the embodiments described herein include but are not limited to Eimeria oocysts, and are selected from the group infecting chickens consisting of E. maxima oocysts, E. mitis oocysts, E. tenella oocysts, E. acervulina oocysts, E. brunetti oocysts, E. necatrix oocysts, E. praecox oocysts, E. hagani oocysts, E. mivati oocysts, and any combination thereof; Eimeria oocysts selected from the group infecting turkeys consisting of E. meleagrimitis oocysts, E. adenoides oocysts, E. gallopavonis oocysts, E. dispersa oocysts, E. innocua oocysts, E. meleagridis oocysts, and E. subrotunda oocysts, and any combination thereof; Eimeria oocysts selected from the group infecting cattle consisting of E. zuernii oocysts, E. bovis oocysts, E. ellipsoidalis oocysts, and any combination thereof; Eimeria oocysts selected from the group consisting of E. ahsata oocysts, E. bakuensis oocysts, E. crandallis oocysts, E. faurei oocysts, E. granulosa oocysts, E. intricata oocysts, E. marsica oocysts, E. ovinoidalis oocysts, E. pallida oocysts, E. parva oocysts, E. weybridgensis oocysts, and any combination thereof; and Eimeria oocysts selected from the group consisting of E. intestinalis oocysts, E. vejdovskyi oocysts, E. piriformis oocysts, E. coecicola oocysts, E. irresidua oocysts, E. flavescens oocysts, E. exigua oocysts, E. magna oocysts, E. perforans oocysts, E. media oocysts, E. stiedae oocysts, and any combination thereof.

The embodiments herein are directed to systems and methods for releasing sporocysts from oocysts. The oocysts can be from a protozoan that infects any animal subject, including mammalian and avian subjects.

Some embodiments described herein may also relate to methods of releasing sporozoites from protozoan oocysts. While this application focuses on Eimeria, some other protozoa form a life stage designated as an “oocyst” but may contain sporozoites within the oocyst and do not produce sporocysts. The embodiments may be practiced to release sporozoites from oocysts of any species of parasite that contains sporozoites within the oocyst, and would include any organisms in the phylum Apicomplexa, and would also include but not be limited to Cryptosporidium and Plasmodium. The terms “protozoa,” “oocyst,” “sporocyst,” “sporozoite” and “merozoite” have their accepted meanings in the art. Unless indicated otherwise, these terms are intended to refer to live (i.e., viable) protozoa, oocysts, sporocysts, sporozoites and merozoites, including wildtype or attenuated forms. Also encompassed herein are genetically modified protozoa, oocysts, sporocysts, sporozoites, and merozoites.

Returning to FIG. 2 and the first embodiment 10, a first reservoir inlet 16 is fixed to the top of the first reservoir 12. The first reservoir inlet 16 receives the solution 14 into the first reservoir 12. A first reservoir outlet 18 is fixed to the first reservoir 12. The first reservoir outlet 18 is connected to a system pump 20. The pump 20 is designed to move the solution 14 from the first reservoir 12 and through the system.

A pump outlet 22 is fixed between the pump 20 and a processing system 24. The processing system 24 of the first embodiment 10 is a high-pressure homogenizer 32. The homogenizer 32 includes a high-pressure source 26, controlled by pressure valve 28 and at least one orifice 34, shown in FIG. 3. The processing system 24 further includes a processing system outlet 38, shown in FIG. 2. The processing system outlet 38 is located within the processing system at the opposed end to the processing system inlet 36. The processing system outlet 38 is connected to a second reservoir 40 by means of a second reservoir inlet 42. The second reservoir 40 is designed to hold a volume of solution received by way of the second reservoir inlet 42.

The second reservoir 40 also includes a second reservoir outlet 44. The second reservoir outlet 44 is fluidly connected to a delivery device 46. In this first embodiment 10, the delivery device 46 is an atomized sprayer. The delivery device 46 has a delivery inlet 48, a nozzle 50 and an air inlet 54 at the nozzle 50 to atomize a plume of spray during delivery. The air inlet 54 is in fluid connection with an air pressure source 56. Alternatively, the delivery device 46 can be a hydraulic spray nozzle, or the like.

In use, the pump 20 is activated so as to move solution 14 out of the first reservoir 12 by way of the first reservoir outlet 18 and into the processing system 24 by way of the processing system inlet 36. While a pump is used to move the solution, other methods including gravity, valves, air pressure, and other methods could be used for fluid movement in any of the embodiments. High-pressure air supplied by the high-pressure homogenizer 32 is used to move solution 14 through the at least one orifice 34. The high-pressure air is over 500 psi, and preferably in the range of 500 to 6,000 psi.

As the solution 14 moves through the orifice 34 under high pressure, the oocysts are subject to shear force and the oocyst membrane will be disrupted or rupture. After the solution 14 passes through the orifice 34, it exits the homogenizer 32 through outlet 38 and moves through inlet 42 into the second reservoir 40. Each oocyst that has its membrane disrupted or rupture, will result in the release of free sporocysts into a modified solution 52. The modified solution 52 (a combination of released sporocysts and residual oocysts) is temporarily stored in the second reservoir 40 until delivery.

When the modified solution 52 is ready for delivery, the modified solution is pumped from the second reservoir 40 through the second reservoir outlet 44 and into the delivery inlet 48. The modified solution 52 is pumped into the nozzle 50 and mixed with pressurized air from the pressurized air source 56. The pressurized air atomizes the modified solution 52 at the nozzle 50 to deliver a predetermined volume of modified solution in the form of a predetermined spray profile to a specific target on an animal.

In an alternative arrangement, the high-pressure homogenizer 32 may be directly connected to the delivery device 46. In this way, the solution produced by the homogenizer 32 is delivered directly via the delivery device 46 rather than temporarily stored in the second reservoir 40.

It should be noted that the embodiments described herein relating to the disrupting of oocysts are intended to be delivered in real time. Any sporocyst-based manufactured vaccine would require a cryoprotectant formulation to maintain the viability of the sporocysts for long term storage and the implementation of a liquid nitrogen cold chain for delivery. The embodiments described herein relating to the disruption of oocyst membranes to release viable sporocysts do not require cryoprotectant solutions or any other special storage conditions such as cryopreservation.

The spray profile is directed to contact the animal at a predetermined location. The present systems are designed to target the animal's facial mucosa, particularly, the eyes or mouth. See PCT Publication WO 2017/083663A1, Karimpour. However, it is appreciated that the spray profile could be designed to target other parts of the animal's body. While it is recognized that the embodiments described herein may be applicable to all animals, the focus is on the delivery of Eimeria which affects poultry, particularly chicken, and more particularly, day-old hatchlings. The free sporocysts in the modified solution are able to quickly enter into the digestive tract of the hatchling. Sporocysts sprayed into the eyes, nasal passages, or mouth of the bird travel to the digestive tract. Sporocysts that are sprayed onto other parts of the bird's body such as the feathers may enter the digestive tract through preening, including self-preening and preening of other birds.

Proteolytic enzymes in the intestinal tract, including trypsin or chymotrypsin, for example, digest the Stieda body at the tip of the sporocyst allowing excystation of the infective sporozoites to occur. This enables the chick to be quickly infected by the Eimeria and thus develop an immune response.

It should be appreciated that the high-pressure homogenizer 24 (or other processing system described in detail below) does not have to be connected to the system, allowing for the processing to be performed off-line and delivered to container 40. The processing of the vaccine with high pressure homogenizer 24 or other systems could occur outside of the spray delivery system but in real time coincident with the preparation of the vaccine for delivery at the hatchery.

### i. Second Embodiment—Bead Treatment

A second embodiment 60 of the present invention is shown in FIG. 4. Similar parts to the first embodiment 10 will be shown with similar numerical indices. The second embodiment 60 includes a first reservoir 12 having an inlet 16 and an outlet 18 and holding a volume of solution 14. The second embodiment 60 also includes a pump 20 and a pressure source 26.

The second embodiment 60 further includes a second processing system 62. The second processing system 62 includes a vessel 64 capable of holding a volume of solution 14. The vessel 64 has an inlet 36 and an outlet 38. The vessel 64 also contains a volume of agitators 70, such as glass, ceramic or metal beads or the like. It should be noted that the agitators 70 may be spherical beads, or any other shape and material that, when agitated in a volume have the capability to crush, smash or otherwise disrupt any solution contained within the volume. The vessel 64 is mounted horizontally, vertically or at some angle to enable it to rotate about its axis. The vessel 64 is connected to a spinner 72 that is capable of rotating the vessel 64 on its horizontal axis. The spinner 72 also has a vibration feature that enables it to vibrate or shake the vessel when activated. In use, the solution 14 after being exposed to the agitators in the vessel 64 results in a second modified solution 74 which will be described in more detail below.

The outlet 38 of the second processing system 62 is connected to the second reservoir 40 by means of an inlet 42. As in the first embodiment 10, the second reservoir outlet 44 is connected to the delivery device 46 where it is ultimately delivered to an animal.

The delivery device 46 includes an inlet 48 having a valve to control the flow of a second modified solution 74 into the inlet 48. It should be noted that the delivery device 46 may be altered to change the spray profile to create more or less atomization or provide a steady stream of fluid from the nozzle 50 directly to the animal.

An alternative arrangement to the second embodiment 60 is similar to the alternative arrangement described above relating to the first embodiment 10. It is envisioned that the second embodiment 60 may be designed to deliver the second modified solution 74 directly to the nozzle 50 and thus eliminate the need for the second reservoir 40.

In use, when the pump 20 and pressure source 26 are activated, the pump moves the solution 14 out of the first reservoir 12 by way of the reservoir outlet 18 and into the second processing system 62. The second processing system 62 is spun along its axis by the spinner 72. In addition, the spinner 72 causes the vessel 64 to vibrate and/or shake. The vibration and spinning cause the agitators 70 to hit and bounce off of the interior vessel walls causing at least some of the oocyst membranes in solution to be disrupted and thus release the sporocysts contained therein. The disrupted oocyst membranes and the viable sporocysts in the solution 14 create the second modified solution 74. The resulting second modified solution 74 is pumped from the vessel 64 and into the second reservoir 40 where it is temporarily held until delivery.

When it is time for delivery, the second modified solution 74 is pumped from the second reservoir 40 to the delivery device 46. The second modified solution 74 is pumped from the delivery device inlet 48 and into the nozzle 50 and mixed with pressurized air from the pressure source 56. The pressurized air atomizes the second modified solution 74 at the nozzle 50 to deliver a predetermined volume of modified solution 74 in the form of a predetermined spray profile to a specific target on an animal, in this embodiment a day-old hatchling.

In the alternative arrangement for the second embodiment 60, the second modified solution 74 is pumped directly from the vessel 64 to the nozzle 50 and to the facial mucosa of a day-old hatchling. The recently disrupted oocyst membranes and released sporocysts are ingested by the hatchling and quickly infect the digestive tract. The hatchling is able to promptly develop an immune response to the Eimeria and remain in good health.

### i. Third Embodiment—Ultrasonication

A third embodiment 80 is similar to the first 10 and second 60 embodiments described above, except for a third embodiment processing system 81. The third embodiment 80, shown in FIG. 5 includes a first reservoir 12 and pump 20. The third embodiment 80 further includes an ultrasonic probe 110 within a vessel 112. The ultrasonic probe 110 further includes a power source (not shown). Ultrasonication can occur with a probe placed directly in solution or with an indirect source placed externally to the vessel. The indirect source may be a source such sonication in a water-filled bath. The term ultrasonicator includes ultrasonic probes and indirect sources external to the vessel.

Oocysts in liquid suspension within the solution 14 are passed through the vessel 112 in close proximity to the ultrasonic probe 110 which vibrates when activated. The preferable range of vibration is between about 18 kHz to 1 MHz. The resulting energy imparted to the solution 14 yields cycles of cavitation which disrupt at least some of the oocyst membranes. The vibrational frequency and flow rate through the system are controlled such that oocyst membranes are disrupted while viable sporocysts exit the vessel 112 intact. The resulting third embodiment solution 114 is either delivered directly to the delivery device 46, such as a sprayer, or to a holding container, such as the second reservoir 40 as described above with regard to the first 10 and second 60 embodiments and alternatives thereto.

In the alternative arrangement for the third embodiment 80, the third modified solution 114 is pumped directly from the vessel 112 to the nozzle 50 and to the facial mucosa of a day-old hatchling. The recently released sporocysts and residual oocysts are ingested by the hatchling and quickly infect the digestive tract. The hatchling is able to promptly develop an immune response to the Eimeria and remain in good health.

A variety of commercial vendors offer sonicators with different configurations. Examples of vendors include Qsonica (Newtown, CT) and Hielscher Ultrasonics GMBH (Teltow, Germany).

### i. Fourth Embodiment—Rotor-Stator Mixer

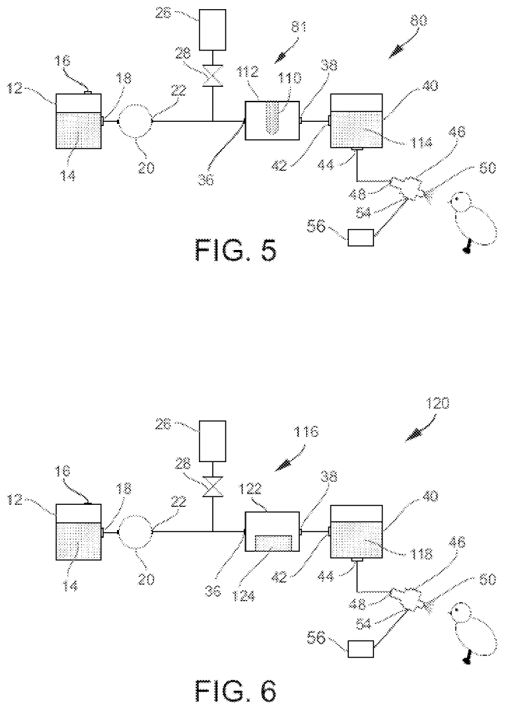

A fourth embodiment 120 uses a rotor/stator mixer processing system 116 within vessel 122 and is shown in FIG. 6. The rotor/stator mixer 116 has a dispersion head or generator 124 therein that rotates at high speed. The fourth embodiment 120 also includes a first reservoir 12, pump 20, and delivery device 46 in fluid communication with the rotor/stator mixer 116.

In use, the rotor/stator mixer 116 is activated which causes the dispersion head or generator 124 to rotate at high speed. The mixer 116 receives the solution 14 from the first reservoir 12. The solution 14 is exposed to the dispersion head or generator 124 rotating at high speed. This causes at least some of oocyst membranes to shear as a result of forces produced within the interior of the mixer 116, thus releasing at least some of the sporocysts. The resulting solution 118 is moved to the delivery device 46 where it is delivered to an animal. As discussed above, an alternative arrangement for the fourth embodiment 120 would include a second reservoir 40 to receive solution 118 which is subsequently delivered to a day-old hatchling.

### i. Fifth Embodiment—Vibrating Plates

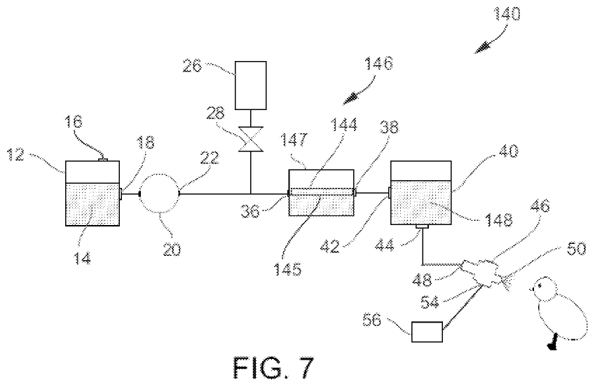

A fifth embodiment 140 is shown in FIG. 7, and is similar to the first 10, second 60, third 80, and fourth 120 embodiments described above except for the processing system 146. The fifth embodiment 140 includes a first reservoir 12 and pump 20. The fifth embodiment 140 includes a fifth processing system 146, within containment 147, made up of a pair of plates 144, 145. The plates 144, 145 are mounted one on top of the other with some space therebetween. The first plate 144 is flat and is connected to a vibration mechanism (not shown). The vibration mechanism causes the plate 144 to vibrate. The second plate 145 may have varying degrees of smoothness or roughness as needed to disrupt the oocyst membranes during their passage between plates 144 and 145. When the plates 144, 145 are on top of each other, second plate 145 makes contact with the first plate 144.

In use, the solution 14 is moved into the fifth processing system 146. A flow of solution 14 is directed between the plates 144, 145. The vibrating mechanism is activated causing the first plate 144 to vibrate against the second plate 145. As the solution 14 moves between the plates 144, 145, at least some of the membranes of the oocysts in solution 14 are disrupted as they pass between the two plates creating a modified solution 148. The modified solution 148 is either delivered directly to the delivery device 46 or to a holding container such as a second reservoir 40.

### a. Sixth Embodiment—Hydrodynamic Cavitation

The sixth embodiment 150 describes a process of vaporization, bubble generation and bubble implosion which occurs in a liquid as the result of a decrease and subsequent increase in local pressure. Cavitation (a phenomenon in which the rapid changes of pressure in a liquid lead to the formation of small vapor-filled cavities that can collapse when subjected to increased pressure) will occur if the pressure declines below the saturation vapor pressure of the liquid and subsequently recovers above the vapor point. This can be produced by passing a liquid through a constricted channel. The process of bubble generation, and the subsequent generation and collapse of the cavitation bubbles, results in high energy densities, and pressure on the surface of the bubbles. In initial studies, cavitation with nitrogen was explored, but any gas could be used to produce similar results. In this example, nitrogen is dissolved in the cytoplasm of the target organism (Eimeria) under pressure. After reaching equilibrium with the environment, the target suspension is abruptly exposed to a change in pressure resulting in nitrogen bubbles forming in the cytoplasm of the Eimeria. The process of intracellular bubble formation and subsequent bubble expansion causes the cellular membrane to stretch and eventually rupture. These bubbles damage the outside of the cell as a result of effervescence (escape of gas from the aqueous solution as the result of a drop in pressure that can result in the creation of foam as well as cell lysis). When tested on a multi-species oocyst suspension of Eimeria, lysis of the oocyst outer wall and subsequent release of the internal sporocysts was observed. This process generally occurs in a pressurized vessel. The vessel consists of a thick stainless-steel casing capable of withstanding high pressure, with an inlet for delivery of gas and an outlet port with an adjustable discharge valve.

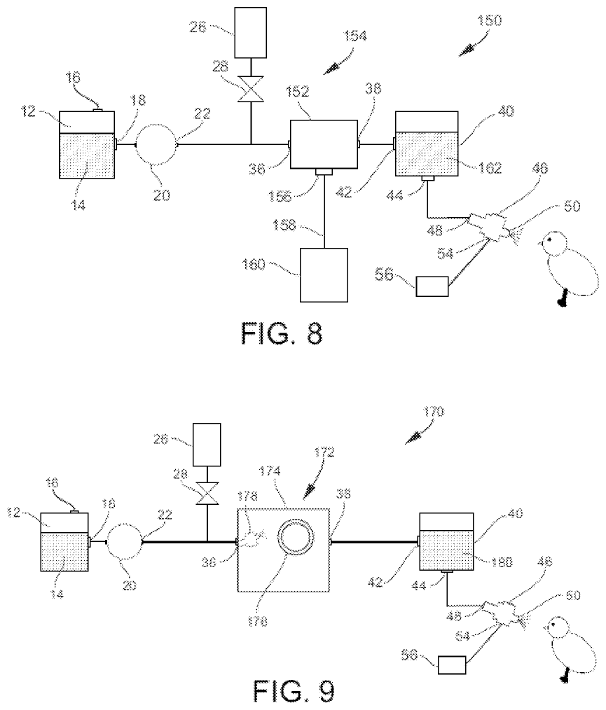

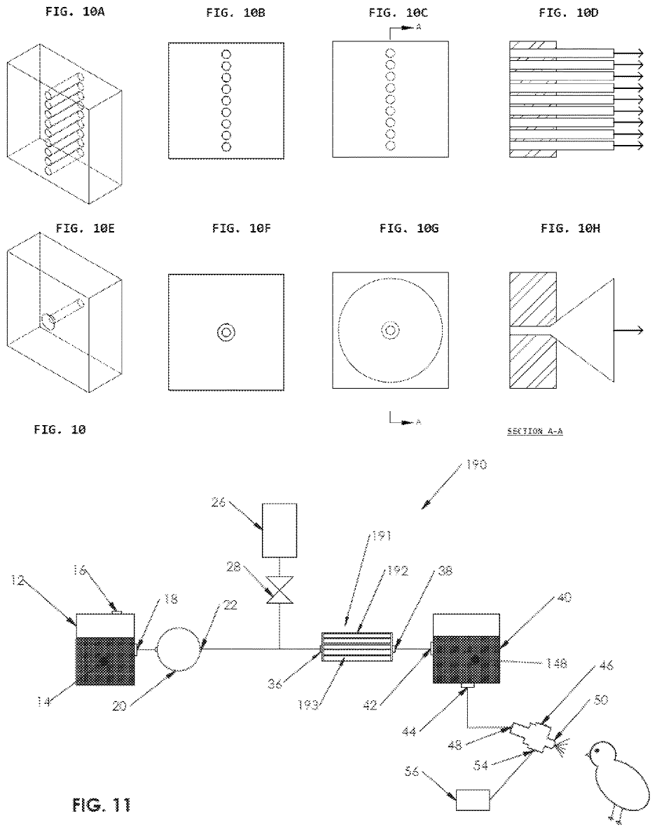

The sixth embodiment 150 is shown in FIG. 8, and is similar to the first 10, second 60, third 80, fourth 120, and fifth 140 embodiments described above except for a sixth processing system 154. The sixth embodiment 150 includes a first reservoir 12 and pump 20. The sixth embodiment 150 includes the sixth processing system 154 made up of a pressurized, metal enclosure 152, and a gas inlet valve 156. The pressurized, metal enclosure 152 is connected to a gas tank source 160 through a filling connector 158. Gas is passed through the filling connector 158 into inlet valve 156, wherein it saturates the aqueous solution present (not shown) within the metal enclosure 154. The solution enters the enclosure via inlet 36 in a manner similar to previous embodiments. Regulators (not shown) are used to control the resulting pressure from the gas flow to achieve optimal saturation.

In use, the solution 14 is moved into the sixth processing system 154. The sixth processing system 154 is then sealed and pressurized. As tank 160 releases nitrogen into the sealed pressurized tank 152, nitrogen is dissolved into the cytoplasm of the oocysts under pressure. After reaching equilibrium with the environment, the flow of nitrogen from tank 160 is ceased and the enclosure 152 is unsealed. Hydrodynamic cavitation occurs within the cytoplasm of the oocysts due to the abrupt change in pressure, causing at least some of the outer membranes to be disrupted, and allowing the internal contents, namely sporocysts, to be released. This creates modified solution 162, which is either delivered directly to the delivery device 46 or to a holding container such as a second reservoir 40.

### a. Seventh Embodiment—High-Pressure Spray

A method for shearing oocysts and releasing intact sporocysts may include spraying a suspension of oocysts at high velocity onto a static object such that the force of impact ruptures the oocyst wall to release viable sporocysts. Additionally, a suspension of oocysts may be sprayed against a moving target, such as a spinning disk so that the combination of forces encountered shear the oocysts. The velocity of the sprayed suspension and the speed of the spinning disk may be adjusted to provide for optimization of the shearing process. The surface features of the spinning disk may be modified to produce varying degrees of smoothness or roughness as needed to shear the oocysts.

A seventh embodiment 170 is shown in FIG. 9, and is similar to the first 10, second 60, third 80, fourth 120, fifth 140, and sixth 150 embodiments described above except for the seventh processing system 172. The seventh embodiment 170 includes a first reservoir 12 and pump 20. The seventh embodiment 170 includes the seventh processing system 172 containing a nozzle 178 or other system to deliver a high-pressure spray or stream of fluid. The fluid will be released from the nozzle and impact on either a solid stationary surface, such as the wall of the vessel 174 or a spinning plate 176.

In use, the solution 14 is moved into the seventh processing system 172. A flow of solution 14 is directed through the nozzle 178. The force of the impact of the solution 14 against the wall of the vessel 174 or the spinning plate 176 will result in disrupting at least some of the oocyst membranes creating a modified solution 180. The modified solution 180 is either delivered directly to the delivery device 46 or to a holding container such as a second reservoir 40.

### a. Additional Embodiments

It is further appreciated that while the embodiments described above often referred to pumps as a means to move solution through the system, it is envisioned that other devices such as high-pressure air and gravitational feed may also be used.

It is further appreciated that while these embodiments have focused on oocyst and sporocyst-based solutions, the solutions described herein may contain other live vaccines including those comprised of viruses, bacteria, yeast, mammalian cells, plant cells, or any genetically modified organisms. It should be apparent to one of skill in the art that solutions containing any such virus, bacteria and the like may be better suited for one embodiment over another based on the particular characteristics of the vaccine.

The system and methods described herein demonstrate that increased efficiency in vaccine response can be achieved using sporocysts, newly released from cracked oocysts and subsequently delivered by spray to initiate the Eimeria infection. A series of experiments using the systems and methods described above have been completed. The results are set forth below. The advantages of generating sporocyst at the time of vaccination and delivering a sporocyst-based vaccine over an oocyst-based vaccine were unexpectedly positive and yielded higher than expected responses in the recipients.

It should be appreciated that while the solution describe herein is the Eimeria oocyst-based vaccine, it is envisioned that there may be other oocyst-based vaccines that could also be delivered using the system and methods described herein. While preferred embodiments primarily relate to Eimeria vaccines for chickens and turkeys, one of ordinary skill would recognize other embodiments relating to Eimeria vaccines for mammals such as cattle, goats, rabbits, or sheep. Moreover, the techniques disclosed herein are useful for improved Apicomplexan vaccines for any species, whether such vaccines are wildtype, or attenuated.

The systems and methods disclosed herein may be adapted for use in aquaculture. Examples of Eimeria which infect fish include, but are not limited to, E. aurati, E. baueri, E. lepidosirenis, E. leucisci, E. rutili, and E. vanasi. Rupturing processes may be applied to these species where applicable to facilitate release of more infective life stages for the purpose of vaccination.

It should also be noted that all embodiments described herein may be applied to an animal individually or en masse. It is appreciated that the embodiments described herein may be applied to a large group of hatchlings, or other animals, contained in a crate or other container and subject to delivery of aqueous solution. The delivery could be in the form of a two-component aqueous solution that forms a gel upon mixing. See PCT application serial number PCT/US19/41178 filed on Jul. 10, 2019 by inventor James Hutchins et al.

a. Eimeria as a Vector to Deliver Recombinant Proteins

The methods and systems disclosed herein may be used with a recombinantly modified Eimeria to serve as vector to deliver other antigen(s). Vaccines for birds or other animals comprising disrupted cellular materials may be administered by the systems and methods described herein, including vaccines originating from viral-infected cells or vaccines originating from cell lines used to produce natural or recombinant protein products or subcellular fragments such as mammalian cells, plant cells, fungal cells, yeast cells, or bacterial cells.

Recent research demonstrates that Eimeria may be successfully transfected and used to express foreign antigens. It could be expected that such antigens could include viral, bacterial, or other antigens for diseases affecting poultry or other proteins or sequences to stimulate the immune system to be used singularly or in combination with antigens. In addition, antigens from other species of Eimeria could be expressed, allowing cross-protection for multiple species of Eimeria to develop from the administration of a single species of Eimeria. For example, Clark et al. and others have shown results demonstrating that Eimeria parasites can be developed as multivalent vaccine vectors and encourage the extension of these studies. See Clark et al., 2012, Eimeria species parasites as novel vaccine delivery vectors: Anti-Campylobacter jejuni protective immunity induced by Eimeria tenella-delivered CjaA, Vaccine 30(16) 2683-2688; Yan et al., 2009, Stable transfection of Eimeria tenella: Constitutive expression of the YFP-YFP molecule throughout the life cycle, Int'l Journal for Parasitology, 39(1) 109-117; and Marugan-Hernandez et al., 2016, Viral proteins expressed in the protozoan parasite Eimeria tenella are detected by the chicken immune system, Parasites & Vectors 9:463 (pub. Aug. 26, 2016, 14 pages). Similarly, Eimeria or other Apicomplexa could be engineered as vectors to deliver other antigens to specific hosts.

The following examples further illustrate the disclosure and are not intended to limit the scope. It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

### EXAMPLES

a. Gavage (Oocysts Vs. Sporocysts)

Oral gavage, delivery via the mouth, is believed to be the gold standard for delivery of oocysts to a bird. In studies using a commercial broiler vaccine, results with oral gavage were found to be more variable than anticipated. Specifically, the results below indicated that infectivity from oocysts delivered by gavage varied greatly by Eimeria species and across test dates. This observation led us to test administration to the eyes (eyedrop) as an alternative delivery means for a positive control. In addition, disrupting the oocysts to release the sporocysts was found to be more effective when delivered via gavage. Oocyst membrane disruption was accomplished via shaking a multi-species oocyst suspension with 4 mm glass beads by a manual process. It may be hypothesized that the day-old hatchlings need feed in their crop to crack the oocysts. In experimental settings and in normal hatchery settings, the hatchlings do not receive food for 3-8 hours due to storage and transportation, allowing the oocysts to pass though the intestinal track unprocessed. Vaccination via eyedrop may slow the movement of the oocysts reaching the gut and could expose the oocysts to different enzymes.

The mechanical disruption of oocyst membranes prior to vaccination, allows for easier processing of the sporocysts, resulting in a greater percentage of the birds being effectively vaccinated, as well as a higher level of oocyst production (output) detected on day 7. This seems to be more critical for the larger species, such as E. maxima, and medium species, such as E. tenella, than the smaller species, e.g., E. acervulina. The smaller species appear to be equally infective when cracked or not. The studies focused efforts on membrane disruption of the large, followed by medium oocysts. Broiler chicks were vaccinated at day of hatch with a 1× dose of commercial vaccine. Intestinal contents were collected from each bird at day 7, and oocysts enumerated via McMaster's chambers by species.

TABLE 1
Comparison of infectivity of gavage with oocysts or sporocysts

| | N | Percent Infected | | |
|---|---|---|---|---|
| Treatment | # birds | E. maxima | E. tenella | E. acervulina |
| Gavage Oocyst | 9 | 70% | 80% | 90% |
| Gavage Oocyst | 15 | 93% | 93% | 100% |
| Gavage Oocyst | 15 | 53% | 53% | 60% |
| Gavage Oocyst | 15 | 60% | 67% | 93% |
| Gavage Oocyst | 15 | 73% | 67% | 100% |
| Gavage Oocyst | 14 | 79% | 93% | 100% |
| Gavage Oocyst | 16 | 25% | 25% | 81% |
| Gavage Oocyst | 15 | 87% | 93% | 100% |
| Gavage Sporocyst | 15 | 100% | 100% | 100% |
| Eyedrop Oocyst | 15 | 80% | 100% | 100% |

Over time, gavage vaccination resulted in inconsistent infectivity as detected by variable results at day 7. More promising results were noted with vaccine processed to release sporocysts as well as with vaccine delivered by eyedrop.

a. Gavage Oocyst Vs Sporocyst

In another experiment, oocyst disruption was accomplished via shaking a multi-species oocyst suspension with 4 mm glass beads by a manual process. Enumeration of the remaining oocysts indicated conversion of approximately 64% of E. maxima oocysts to sporocysts, 51% of E. tenella oocysts to sporocysts and 17% of E. acervulina oocysts to sporocysts. Day of hatch chicks (15 per treatment) were inoculated with either oocyst or glass-bead-released sporocysts with residual oocysts via oral gavage and intestinal contents were collected on day 7.

TABLE 2
Comparison of frequency and amplitude of response with gavage of oocysts
or sporocysts with residual oocysts

| | Frequency of Response | | | Amplitude of Response | | |
|---|---|---|---|---|---|---|
| Oral Gavage | (Birds infected/Birds vaccinated) | | | (Average oocyst output per bird) | | |
| Treatment | E. maxima | E. tenella | E. acervulina | E. maxima | E. tenella | E. acervulina |
| Oocysts | 13/15 (87%) | 14/15 (93%) | 15/15 (100%) | 2.30 × 10⁵ | 4.71 × 10⁵ | 8.94 × 10⁵ |
| Sporocysts | 15/15 (100%) | 15/15 (100%) | 15/15 (100%) | 3.41 × 10⁵ | 8.58 × 10⁵ | 1.08 × 10⁶ |
| Improvement | 13% | 7% | 0% | 1.48-fold | 1.84-fold | 1.21-fold |

The improvement shown above for frequency of response is the difference between the percentage of birds infected with the sporocysts and oocysts treatments. The improvement shown for amplitude of response is the oocyst output per bird for the sporocyst treatment divided by the oocyst output per bird for the oocyst treatment.

In this particular case, both the frequency of response and amplitude of response were increased in chicks orally vaccinated with sporocyst vaccine containing residual oocysts as compared to oocyst-based vaccine.

The variable rates of infectivity observed for gavaged birds raises questions about the effectiveness of oocyst-based vaccines administered at the hatchery. Birds administered oocyst-based vaccines at the hatchery lack the presence of food and grit in the digestive tract which may be particularly critical for processing the E. maxima oocysts to the sporocyst stage. If so, these birds would be at risk for low levels of E. maxima infectivity during the first round of infection. It would follow, then, that during the second round of infection, the largely naïve population would run the risk of extremely high infection and output rates, with additional risk of secondary bacterial infections in damaged gut tissues. Indeed, this situation is often the case, and is one of the primary reasons some avoid vaccination and use anticoccidials and chemicals in the feed instead. The use of sporocyst-based vaccine administered by eye spray at the hatchery has the potential to markedly improve infectivity of E. maxima during the first round of infection, and thus avoid severe coccidiosis infections and risk of secondary infections during the second round of infection at a grow out facility.

a. Eyedrop Oocyst Vs Sporocyst

In another experiment, oocyst membrane disruption was completed via shaking a multi-species oocyst suspension with 4 mm glass beads by a manual process. Enumeration of the remaining oocysts indicated conversion of approximately 52% of E. maxima oocysts to sporocysts, 26% of E. tenella oocysts to sporocysts and 47% of E. acervulina oocysts to sporocysts. Day of hatch chicks (15 per treatment) were inoculated with either oocysts or glass-bead-released sporocysts and residual oocysts via eyedrop and intestinal contents were collected on day 7.

TABLE 3
Comparison of frequency and amplitude of response with eyedrop
administration of oocysts or sporocyst vaccine containing residual oocysts

| | Frequency of Response | | | Amplitude of Response | | |
|---|---|---|---|---|---|---|
| Eyedrop | (Birds infected/Birds vaccinate) | | | (Average oocyst output per bird) | | |
| Treatment | E. maxima | E. tenella | E. acervulina | E. maxima | E. tenella | E. acervulina |
| Oocysts | 11/14 (79%) | 11/14 (79%) | 14/14 (100%) | 2.82 × 10⁵ | 7.89 × 10⁴ | 7.60 × 10⁵ |
| Sporocysts | 15/15 (100%) | 15/15 (100%) | 15/15 (100%) | 7.14 × 10⁵ | 3.06 × 10⁵ | 5.66 × 10⁵ |
| Improvement | 21% | 21% | 0% | 2.5-fold | 3.9-fold | none |

It was found that the sporocyst treatment group yielded improved results in both frequency and amplitude of response for E. maxima and E. tenella. No oocysts were observed in the intestinal contents of the untreated control birds.

a. Delivery of Sporocysts by Spray

In the following set of experiments, release of sporocysts was achieved by shaking a multi-species oocyst suspension with 4 mm glass beads by hand. By enumeration of the remaining oocysts, the process was calculated to have converted at a minimum 66% of large oocysts (E. maxima species) to sporocysts, 75% of medium oocysts (E. tenella species) to sporocysts, and 13% of small oocysts (E. acervulina and other small species) to sporocysts. Day of hatch chicks (15 per treatment) were held in a stationary position and sprayed from a device with either the untreated oocyst or glass-bead-released sporocyst treatments, as previously described above. The spray was administered through an air atomizing nozzle utilizing both liquid and air pressure aimed at the facial mucosa of the chick. The birds were then given access to food and water, grown for 7 days and then sacrificed to collect each bird's intestinal contents. Below are the data tables associated with these respective spray vaccination experiments.

TABLE 4
Frequency and amplitude of response for birds sprayed with oocysts or
sporocysts for Study A

| | Frequency of Response | | | Amplitude of Response | | |
|---|---|---|---|---|---|---|
| | (Birds infected/Birds vaccinated) | | | (Average oocyst output per bird) | | |
| Treatment | E. maxima | E. tenella | E. acervulina | E. maxima | E. tenella | E. acervulina |
| Oocysts | 7/15 (47%) | 11/15 (73%) | 13/15 (87%) | 2.74 × 10⁴ | 2.40 × 10⁴ | 5.66 × 10⁴ |
| Sporocysts | 14/15 (93%) | 14/15 (93%) | 15/15 (100%) | 2.55 × 10⁵ | 1.84 × 10⁵ | 2.9 × 10⁵ |
| Improvement | 46% | 20% | 13% | 9.7-fold | 7.7-fold | 5.1-fold |

TABLE 5
Frequency and amplitude of response for birds sprayed with oocysts or
sporocysts for Study B

| | Frequency of Response | | | Amplitude of Response | | |
|---|---|---|---|---|---|---|
| | (Birds infected/Birds vaccinated) | | | (Average oocyst output per bird) | | |
| Treatment | E. maxima | E. tenella | E. acervulina | E. maxima | E. tenella | E. acervulina |
| Oocysts | 5/15 (33%) | 9/15 (60%) | 15/15 (100%) | 7.39 × 10⁴ | 2.70 × 10⁴ | 7.32 × 10⁴ |
| Sporocysts | 12/15 (80%) | 13/15 (87%) | 15/15 (100%) | 1.37 × 10⁵ | 1.46 × 10⁵ | 1.39 × 10⁵ |
| Improvement | 47% | 27% | 0% | 1.85-fold | 5.4-fold | 1.9-fold |

TABLE 6
Frequency and amplitude of response for birds sprayed with oocysts or
sporocysts for Study C

| | Frequency of Response | | | Amplitude of Response | | |
|---|---|---|---|---|---|---|
| Spray | (Birds infected/Birds vaccinated) | | | (Average oocyst output per bird) | | |
| Treatment | E. maxima | E. tenella | E. acervulina | E. maxima | E. tenella | E. acervulina |
| Oocysts | 10/15 (67%) | 11/15 (73%) | 13/15 (87%) | 1.10 × 10⁵ | 2.27 × 10⁵ | 1.60 × 10⁵ |
| Sporocysts | 14/15 (93%) | 12/15 (80%) | 14/15 (93%) | 4.31 × 10⁵ | 7.25 × 10⁵ | 1.72 × 10⁵ |
| Improvement | 27% | 7% | 6% | 3.9-fold | 3.2-fold | 1.1-fold |

It was found by oocyst enumeration that the frequency of infection and the amplitude of response were both improved with the sporocyst treatment.

Across all three experiments, no oocysts were observed in the intestinal contents of the untreated control birds, and positive controls, inoculated via eyedrop, yielded infectivity frequencies and amplitudes higher than those of the experimental spray treatment groups.

It should be appreciated that the results set forth in Tables 4, 5 and 6 were unexpected. The results indicate that a higher than expected uptake of the vaccine through sporocyst vaccination occurred in the chicks. This is believed to be attributed, in part, to the chick's inability to properly process unbroken oocysts in the digestive tract. The infection by sporocysts in the chicks indicates that a significant number of chicks are able to process and become infected with the sporocyst but not the oocyst. The effect is seen more for E. maxima and E. tenella than with E. acervulina. Thus, it is believed that due to absence of abrasive material in a chick's upper digestive system, including the crop and the gizzard, the E. maxima and E. tenella oocysts are not easily broken. Sporocysts do not require abrasive material in a chick's digestive system for excystation of sporozoites. Sporocysts are easily processed to the infective sporozoite life stage by enzymes, such as proteases, in the digestive tract.

In another experiment, where birds were vaccinated in a stationary setting, grown and sacrificed at day 7 to collect intestinal contents, the effects of sporocyst versus oocyst inoculation were seen. In this case however, the chicks were sprayed with a nozzle solely utilizing liquid pressure as opposed to both liquid and air pressure. The lack of air pressure created a stream-like fluid dispense as compared to an atomized cone of vaccine. The results from this experiment can be seen in the table below.

TABLE 7
Frequency and amplitude of response for birds sprayed with oocysts or
sporocysts without air pressure

| | Frequency of Response | | | Amplitude of Response | | |
|---|---|---|---|---|---|---|
| | (Birds infected/Birds vaccinated) | | | (Average oocyst output per bird) | | |
| Treatment | E. maxima | E. tenella | E. acervulina | E. maxima | E. tenella | E. acervulina |
| Oocysts | 53/60 (88%) | 58/60 (97%) | 59/60 (98%) | 1.31 × 10⁵ | 4.37 × 10⁵ | 4.74 × 10⁵ |
| Sporocysts | 15/15 (100%) | 15/15 (100%) | 15/15 (100%) | 3.80 × 10⁵ | 4.78 × 10⁵ | 6.63 × 10⁵ |
| Improvement | 12% | 3% | 7% | 2.91-fold | 1.09-fold | 1.40-fold |

No oocysts were observed in the intestinal contents of the untreated control birds, and positive controls, inoculated via eyedrop, yielded infectivity frequencies and amplitudes higher than those of the experimental spray treatment groups. The frequency of response and amplitude of response were numerically larger for E. maxima and to a lesser extent E. tenella and E. acervulina. These results indicate that regardless of the spray pattern from a nozzle, the administration of a sporocyst with residual oocyst solution yields improved results as compared to those of an oocyst solution.

In addition to manual processing of oocysts to release sporocysts, an automated process using an IKA Ultra Turrax device (IKAR-Werke Gmbh & Co., Staufen, Germany) with glass beads. Glass beads were added to the vaccine volume (borosilicate glass balls, size 1-6 mm) and processed for 20-240 seconds at 4,000-8,000 rpm. Results for oocysts shearing were equivalent to manual processes with improved repeatability. Enumeration of the remaining oocyst after being processed by the IKA UTTD device indicated conversion of approximately 76% of E. maxima oocysts to sporocysts, 70% of E. tenella oocysts to sporocysts and 46% of E. acervulina oocysts to sporocysts. An experiment using broiler chicks with the same methodology as aforementioned yielded the following results.

TABLE 8
Frequency and amplitude of response for birds sprayed with oocysts or
sporocysts with residual oocysts processed by the IKA UTTD device

| | Frequency of Response | | | Amplitude of Response | | |
|---|---|---|---|---|---|---|
| | (Birds infected/Birds vaccinated) | | | (Average oocyst output per bird) | | |
| Treatment | E. maxima | E. tenella | E. acervulina | E. maxima | E. tenella | E. acervulina |
| Oocysts | 5/15 (33%) | 4/15 (27%) | 13/15 (87%) | 2.3 × 10⁴ | 4.16 × 10³ | 8.91 × 10⁴ |
| Sporocysts | 11/14 (79%) | 11/14 (79%) | 12/14 (86%) | 2.83 × 10⁵ | 2.70 × 10⁵ | 1.2 × 10⁵ |
| Improvement | 46% | 52% | 0% | 12.3-fold | 64.8-fold | 1.2-fold |

No oocysts were observed in the intestinal contents of the untreated control birds, and positive controls, inoculated via eyedrop, yielded infectivity frequencies and amplitudes higher than those of the experimental spray treatment groups. The frequency of response and amplitude of response were numerically larger for E. maxima and E. tenella, and to a lesser extent E. acervulina. This data suggests that the IKA UTTD device can shear oocyst to generate a sporocyst/residual oocyst solution and provide similar results to that of hand shaking process. In yet another experiment, the differences between vaccinating with sporocyst versus oocyst can be seen. In this experiment, the release of sporocysts was achieved by shaking a multi-species oocyst suspension with 4 mm glass beads by hand. This sporocyst/residual oocyst solution and oocyst-only solution were administered to a day of hatch aimed at their facial mucosa. Two sets of nozzles were employed, with the first set administering a 2% sodium alginate solution and the second set administering vaccine in 3.0% calcium chloride solution. When these two solutions come into contact on the surface of the bird a gel is formed. The creation of gel is hypothesized to keep the oocyst/sporocyst vaccine hydrated longer as compared to a typical aqueous spray, extending the potential preening time for the birds. See PCT No. PCT/US19/41178, Hutchins et al., filed Jul. 10, 2019, Atty. Dkt. 673-04-PCT. The results from this experiment can be seen in the table below.

TABLE 9
Frequency and amplitude of response for birds sprayed with oocysts or
sporocysts with residual oocysts in a gel

| | Frequency of Response | | | Amplitude of Response | | |
|---|---|---|---|---|---|---|
| Gel Spray | (Birds infected/Birds vaccinated) | | | (Average oocyst output per bird) | | |
| Treatment | E. maxima | E. tenella | E. acervulina | E. maxima | E. tenella | E. acervulina |
| Oocysts | 6/15 (40%) | 7/15 (47%) | 13/15 (87%) | 7.67 × 10⁴ | 1.26 × 10⁵ | 8.46 × 10⁴ |
| Sporocysts | 15/15 (100%) | 15/15 (100%) | 15/15 (100%) | 2.51 × 10⁵ | 4.83 × 10⁵ | 1.90 × 10⁵ |
| Improvement | 60% | 53% | 13% | 3.3-fold | 3.8-fold | 2.24-fold |

No oocysts were observed in the intestinal contents of the untreated control birds, and positive controls, inoculated via eyedrop, yielded infectivity frequencies and amplitudes higher than those of the experimental spray treatment groups. These results indicate that gel formulation sporocyst outperformed oocyst only in both frequency and amplitude of response. It has been noted that the infectivity of the smaller E. acervulina species is relatively high even if administered as oocysts rather than sporocysts. The infectivity data would indicate that E. acervulina oocysts can be processed to the sporocyst life stage and then to the sporozoite life stage efficiently in the digestive tract of the chicken whether feed and grit are present or not.

Gavage infectivity evidence would indicate that E. maxima especially may not be processed in vivo to the sporocyst stage without feed or grit being present in the digestive tract. Infectivity from E. maxima, and to a somewhat lesser extent, E. tenella is boosted by pre-processing the oocyst life stage to the sporocyst life stage prior to administration. In vitro, however, the shear force required to crack the larger E. maxima oocysts is much less than the shear force required to crack the smaller E. acervulina oocysts. (European Patent 2,111, 243 B1 (Hutchins et al., Embrex, Inc.)). Therefore, a system for producing sporocyst at the point of use provides a complimentary action, combining the in vitro efficiency of cracking the larger oocyst species with the in vivo efficiency of processing the smaller oocyst species to yield more robust vaccine efficacy.

a. Results from a High-Pressure Homogenizer
i. Oocyst Reduction Counts—In Vitro

A liquid suspension containing oocysts of mixed Eimeria species was processed through a high-pressure homogenizer (HPH) IKA model 2000-4 at varying pressures. The cell suspension was loaded into the inlet of the HPH, processed at a range of pressures (200-1500 bar), and then dispensed from an outlet. The intact oocysts contained in the liquid preparation were enumerated using McMaster's floatation chambers before and after exposure to the HPH. Results are shown in the table below.

TABLE 10
A comparison of residual oocyst processed at different
pressures with high-pressure homogenization (HPH)

| | | | | Percent oocyst reduction compared to | | | |
|---|---|---|---|---|---|---|---|
| | Total Oocysts per | | | starting material | | | |
| | Sample (Average) | | | | | | Total |
| HPH | E. maxima | E. tenella | E. acervulina | E. maxima | E. tenella | E. acervulina | Oocysts |
| Original | 1.74 × 10² | 8.31 × 10¹ | 5.46 × 10² | — | — | — | — |
| material | | | | | | | |
| 200 bar | 7.47 × 10¹ | 4.2 × 10¹ | 4.48 × 10² | 57% | 49% | 18% | 30% |
| 500 bar | 4.67 × 10⁰ | 4.20 × 10¹ | 2.99 × 10² | 97% | 49% | 45% | 73% |

Results showed that the higher the pressure, the higher the percent oocyst reduction, and therefore the higher the percent sporocyst release was obtained.

Frequency and amplitude of response for birds sprayed with oocysts or sporocysts—In vivo

In this experiment birds were either exposed to a nozzle spray consisting of an oocyst solution or a sporocyst solution with residual oocysts processed by the IKA HPH 2000-4 device. The birds were then provided with food and water, grown and sacrificed at day 7 to collected individual intestinal contents. The results from this experiment are displayed in the tables below.

TABLE 11
A comparison of infectivity of unprocessed oocysts versus
oocysts processed by IKA HPH 2000-4 at 200 bar

| | Frequency of Response | | | Amplitude of Response | | |
|---|---|---|---|---|---|---|
| | (Birds infected/Birds sprayed) | | | (Average oocyst output per bird) | | |
| Treatment | E. maxima | E. tenella | E. acervulina | E. maxima | E. tenella | E. acervulina |
| Oocysts | 7/15 (47%) | 6/15 (40%) | 15/15 (100%) | 8.42 × 10⁴ | 5.09 × 10³ | 8.1 × 10⁴ |
| 200 bar | 8/14 (57%) | 1/14 (7%) | 7/14 (50%) | 1.26 × 10⁴ | 1.17 × 10⁴ | 1.31 × 10⁴ |
| Improvement | 10% | 0% | 0% | none | 2.29-fold | none |

TABLE 12
A comparison of infectivity of unprocessed oocysts versus
oocysts processed by IKA HPH 2000-4 at 500 bar

| | Frequency of Response | | | Amplitude of Response | | |
|---|---|---|---|---|---|---|
| | (Birds infected/Birds sprayed) | | | (Average oocyst output per bird) | | |
| Treatment | E. maxima | E. tenella | E. acervulina | E. maxima | E. tenella | E. acervulina |
| Oocysts | 7/15 (47%) | 6/15 (40%) | 15/15 (100%) | 8.42 × 10⁴ | 5.09 × 10³ | 8.1 × 10⁴ |
| 500 bar | 3/15 (20%) | 7/15 (47%) | 13/15 (87%) | 2.23 × 10³ | 1.05 × 10⁵ | 3.63 × 10⁴ |
| Improvement | 0% | 7% | 0% | none | 20.66-fold | none |

No oocysts from any species were observed in the intestinal contents of the 15 untreated control birds. The positive controls inoculated via eye drop yielded infectivity frequencies of 100% (across all species) and higher amplitudes (across all species) as compared to the experimental treatment groups. While the oocyst reduction percentages for the IKA HPH looked satisfactory from the in vitro data, it is hypothesized that the sporocysts generated from the homogenization process were also damaged, therefore resulting in poor frequency and amplitude responses in the in vivo data.

a. Results from a Rotor-Stator-Like Device
i. Oocyst Reduction Counts—In Vitro

A liquid oocyst suspension containing mixed Eimeria species was processed at varying speeds through the IKA Magic Lab, a single pass, inline, rotor-stator-like device. The cell suspension was loaded into the hopper of the Magic Lab, processed through 1-3 rotor stator generators, at varying speeds (3,000-26,000 rpm) and then dispensed from an outlet. The generators used for this experiment were the 6F model. Intact oocysts in the liquid preparation were enumerated using McMaster's floatation chambers before and after processing with the Magic Lab device. Results are shown in the table below:

TABLE 13
A comparison of residual oocysts processed at different speeds with rotor-stator

| | | | | Percent reduction compared to | | | |
|---|---|---|---|---|---|---|---|
| | Total Oocyst per | | | starting material | | | |
| | Sample (Average) | | | | | | Total |
| Rotor-stator | E. maxima | E. tenella | E. acervulina | E. maxima | E. tenella | E. acervulina | Oocyst |
| Original material | 1.86 × 10² | 8.87 × 10¹ | 5.22 × 10² | — | — | — | — |
| 16,000 RPM | 1.26 × 10² | 5.13 × 10¹ | 5.04 × 10² | 32% | 42% | 3% | 14% |
| 26,000 RPM | 1.03 × 10² | 8.4 × 10¹ | 5.09 × 10² | 45% | 5% | 2% | 13% |

Oocyst reduction was consistently observed for E. maxima, while variable reduction was observed for E. tenella and minimal reduction observed for E. acervulina.

Frequency and amplitude of response for birds sprayed with oocysts or sporocysts—In vivo

In this experiment, day of hatch broiler chicks were either exposed to a nozzle spray consisting of an oocyst solution or a sporocyst solution with residual oocysts processed by the IKA Magic Lab device. The birds were then provided with food and water, grown and sacrificed at day 7 to collected individual intestinal contents. The results from this experiment are displayed in the tables below.

TABLE 14
A comparison of infectivity of unprocessed oocysts versus
oocysts processed by IKA Magic Lab at 16,000 rpm

| | Frequency of Response | | | Amplitude of Response | | |
|---|---|---|---|---|---|---|
| | (Birds infected/Birds sprayed) | | | (Average oocyst output per bird) | | |
| Treatment | E. maxima | E. tenella | E. acervulina | E. maxima | E. tenella | E. acervulina |
| Oocysts | 7/15 (47%) | 6/15 (40%) | 15/15 (100%) | 8.42 × 10⁴ | 5.09 × 10³ | 8.1 × 10⁴ |
| 16,000 rpm | 9/15 (60%) | 4/15 (27%) | 14/15 (93%) | 5.26 × 10⁴ | 1.05 × 10³ | 1.12 × 10⁵ |
| Improvement | 13% | 0% | 0% | None | None | 1.47-fold |

TABLE 15
A comparison of infectivity of unprocessed oocysts versus
oocysts processed by IKA Magic Lab at 26,000 rpm

| | Frequency of Response | | | Amplitude of Response | | |
|---|---|---|---|---|---|---|
| | (Birds mfected/Birds sprayed) | | | (Average oocyst output per bird) | | |
| Treatment | E. maxima | E. tenella | E. acervulina | E. maxima | E. tenella | E. acervulina |
| Oocysts | 7/15 (47%) | 6/15 (40%) | 15/15 (100%) | 8.42 × 10⁴ | 5.09 × 10³ | 8.1 × 10⁴ |
| 26,000 rpm | 9/15 (60%) | 11/15 (73%) | 14/15 (93%) | 5.68 × 10⁴ | 3.42 × 10⁵ | 1.22 × 10⁵ |
| Improvement | 13% | 33% | 0% | None | 67-fold | 1.51-fold |

No oocysts from any species were observed in the intestinal contents of the 15 untreated control birds. The positive controls inoculated via eye drop yielded infectivity frequencies of 100% (across all species) and higher amplitudes (across all species) as compared to the experimental treatment groups. The frequency of response was increased for E. maxima in both Magic Lab treatment groups however, the amplitude of response for those respective treatment groups were less than those belonging to the oocyst treatment group, but still within an acceptable range. The frequency and amplitude of response were most notably increased for E. tenella. These results may indicate that E. maxima oocysts were over processed under the conditions used.

a. Hydrodynamic Cavitation Experiments
i. In Vitro Studies with Hydrodynamic Cavitation

For initial testing, a low cost, leak-free reinforced aluminum whipped cream dispenser (EurKitchen EK-WHIP-18) with an attachment for a nitrogen charger and an outlet were used. The liquid containing a mixed Eimeria preparation was loaded into the canister, the nitrogen gas cartridge was added, and the canister was then inverted several times to allow the gas to saturate the liquid. The canister was turned upside down and the valve opened to create a pressure drop and release the liquid. The liquid preparation was counted before and after exposure to hydrodynamic cavitation to determine the percentage of oocyst disrupted by species. In some cases, the liquid was processed and released from the system, loaded back into the system, and then re-exposed to hydrodynamic cavitation two or three more times to disrupt more cells, as pressure and amount of gas could not be controlled in the initial system tests.

For the purposes of this disclosure, the act of releasing liquid that has already undergone hydrodynamic cavitation, and then reloading that same liquid back into the same vessel to be re-exposed to hydrodynamic cavitation, will be referred to as a “Pass.” As such, from this point onward the terminology of “Pass 1,” “Pass 2,” or “Pass 3” will be used to represent the act of exposing a liquid to the process of hydrodynamic cavitation a set number of times. The number of times will be defined by the number following “Pass.”

TABLE 16
A comparison of residual oocysts processed with multiple
cycles of whipped cream dispenser hydrodynamic cavitation

| | Total Oocysts per | | | Percent reduction compared to | | | |
|---|---|---|---|---|---|---|---|
| Hydrodynamic | Sample (Average) | | | starting material | | | |
| Cavitation | E. maxima | E. tenella | E. acervulina | E. maxima | E. tenella | E. acervulina | Total |

Experiment 1

| | | | | | | | |
|---|---|---|---|---|---|---|---|
| Original material | 4.15 × 10² | 1.63 × 10² | 1.19 × 10³ | — | — | — | — |
| Pass 1 | 2.88 × 10² | 9.8 × 10¹ | 8.7 × 10² | 31% | 40% | 30% | 31% |

Experiment 2

| | | | | | | | |
|---|---|---|---|---|---|---|---|
| Original material | 3.03 × 10² | 2.12 × 10² | 7.63 × 10² | — | — | — | — |
| Pass 1 | 2.05 × 10² | 1.14 × 10² | 6.14 × 10² | 32% | 46% | 20% | 27% |
| Pass 2 | 1.62 × 10² | 5.83 × 10¹ | 3.56 × 10² | 47% | 73% | 53% | 55% |
| Pass 3 | 1.70 × 10² | 6.65 × 10¹ | 4.35 × 10² | 44% | 69% | 43% | 47% |

Experiment 1 (see table 16) was performed to gauge the worth of pursuing a new cell disruption technique, and Experiment 2 was performed to further explore the technique. Ultimately, Experiment 1 showed comparable conversion of oocysts to sporocysts across all species that performed similarly to early tests with previously explored oocyst shearing techniques, such as shaking with glass beads. In Experiment 2 “Pass 2” demonstrated superior conversion to “Pass 1,” while “Pass 3” showed comparable results to “Pass 2,” indicating that increased exposure to nitrogen cavitation did not necessarily improve the conversion further.

Following the success of hydrodynamic cavitation under the limited parameters of the previously established EurKitchen approach, the protocol was refined further to incorporate a commercial grade hydrodynamic cell disruptor, the process for which is explored in greater detail in the sixth embodiment.

Further investigations were performed by using a cell disruption vessel (Parr™ 4639) with nitrogen gas supplied through a nitrogen filling connection (Parr™ 1831) at varying pressures, 1000 psi and 1500 psi, and then allowing the nitrogen gas to dissolve into the dilute vaccine for 5 minutes.

Although specifics are provided for pressure and time, it should be stated that the specifics of the process can occur across a broader range. This range can consist of pressures ranging from 500-5000 psi, time of exposures that last from 1-30 minutes and diluents that vary in composition. These compositions can consist of distilled water, phosphate buffered saline (PBS) and other variants of the two.

TABLE 17
in vitro comparison of residual oocysts processed by multiple
pressures of cell disruption vessel hydrodynamic cavitation

| | | | | Percent reduction compared to | | | |
|---|---|---|---|---|---|---|---|
| | Total Oocysts per | | | respective starting material | | | |
| Hydrodynamic | Sample (Average) | | | | | | Total |
| Cavitation | Large | Medium | Small | Large | Medium | Small | Oocyst |
| Original material | 2.22 × 10² | 1.12 × 10² | 4.06 × 10² | — | — | — | — |
| (1000 psi) | | | | | | | |
| 1000 psi/5 min | 9.10 × 10¹ | 6.30 × 10¹ | 3.34 × 10² | 47% | 5% | 23% | 27% |
| Original material | 2.59 × 10² | 1.54 × 10² | 4.48 × 10² | — | — | — | — |
| (1500 psi) | | | | | | | |
| 1500 psi/5 min | 1.14 × 10² | 8.17 × 10¹ | 3.22 × 10² | 56% | 47% | 28% | 40% |

The liquid preparation was enumerated via the McMaster's floatation chamber method before and after being processed by hydrodynamic cavitation. The results showed that both tested pressures were able to convert approximately 50% and 25% of oocysts to sporocysts in the large and small species respectively. There was an increased conversion in the medium species at the higher tested pressure.

i. In Vivo Hydrodynamic Cavitation Studies (1^{st} Method)

In this series of experiments, oocyst membrane disruption was completed using hydrodynamic cavitation and infectivity was evaluated via an oocyst output model. Enumeration of the remaining oocysts were calculated and shown below. Day of hatch chicks (15 per treatment) were inoculated with either oocysts or hydrodynamic cavitation-released sporocysts with residual oocysts via spray vaccination and intestinal contents were collected on day 7.

The data in Tables 18 & 19 below demonstrate the effectiveness of in vivo delivery of EurKitchen hydrodynamic cavitation-released sporocysts. The data in Table 19 was collected in a separate experiment from the data in Table 18.

TABLE 18
In vivo infectivity comparison of multiple cycles of artisan
whipped cream dispenser hydrodynamic cavitation

| | Frequency of Response | | | Amplitude of Response | | |
|---|---|---|---|---|---|---|
| Hydrodynamic | (Birds infected/Birds sprayed) | | | (Average oocyst output per bird) | | |
| Cavitation | E. maxima | E. tenella | E. acervulina | E. maxima | E. tenella | E. acervulina |
| Pass 1 | 6/15 (40%) | 8/15 (53%) | 9/15 (60%) | 5.77 × 10⁴ | 1.44 × 10⁵ | 1.35 × 10⁵ |
| Pass 2 | 9/15 (60%) | 5/15 (33%) | 13/15 (87%) | 2.08 × 10⁴ | 1.13 × 10³ | 1.36 × 10⁴ |
| Pass 3 | 6/15 (40%) | 4/15 (27%) | 12/15 (80%) | 8.23 × 10³ | 1.45 × 10⁵ | 3.00 × 10⁴ |

TABLE 19
In vivo infectivity comparison of artisan whipped cream dispenser hydrodynamic cavitation

| | Frequency of Response | | | Amplitude of Response | | |
|---|---|---|---|---|---|---|
| Hydrodynamic | (Birds infected/Birds sprayed) | | | (Average oocyst output per bird) | | |
| Cavitation | E. maxima | E. tenella | E. acervulina | E. maxima | E. tenella | E. acervulina |
| Oocysts | 10/12 (83%) | 10/12 (83%) | 12/12 (100%) | 5.23 × 10⁵ | 6.84 × 10⁴ | 3.71 × 10⁵ |
| Pass 2 | 14/15 (93%) | 14/15 (93%) | 15/15 (100%) | 4.13 × 10⁵ | 4.21 × 10⁵ | 7.2.6 × 10⁵ |
| Improvement | 10% | 10% | 0% | none | 6.2-fold | 2.0-fold |

No oocysts were observed in the intestinal contents of the untreated control birds. The eye drop control for this experiment yielded higher frequency and amplitude of response as compared to the experimental treatment groups. Table 18 demonstrated the comparative effectiveness of different “Pass” numbers and Table 19 demonstrated the effectiveness of the best performing, “Pass 2” compared to an oocyst eye spray delivery.

i. In Vivo Hydrodynamic Cavitation Studies (2nd Method)

The data in Tables 20 & 21 below demonstrate the effectiveness of the cell disruption process via cell disruption vessel hydrodynamic cavitation. The data in both Tables 20 & 21 were collected from the same experiment.

TABLE 20
in vivo infectivity comparison of cell disruption vessel hydrodynamic cavitation (1000 psi)

| | Frequency of Response | | | Amplitude of Response | | |
|---|---|---|---|---|---|---|
| Hydrodynamic | (Birds infected/Birds sprayed) | | | (Average oocyst output per bird) | | |
| Cavitation | E. maxima | E. tenella | E. acervulina | E. maxima | E. tenella | E. acervulina |
| Oocysts | 7/15 (47%) | 5/15 (40%) | 15/15 (100%) | 8.42 × 10⁴ | 5.09 × 10³ | 8.10 × 10⁴ |
| 1000 psi | 12/15 (80%) | 9/15 (60%) | 13/15 (87%) | 3.38 × 10⁵ | 2.38 × 10⁵ | 1.11 × 10⁵ |
| Improvement | 33% | 20% | 0% | 4.0-fold | 46.6-fold | 1.4-fold |

TABLE 21
In vivo infectivity comparison of cell disruption vessel hydrodynamic cavitation (1500 psi)

| | Frequency of Response | | | Amplitude of Response | | |
|---|---|---|---|---|---|---|
| Hydrodynamic | (Birds infected/Birds sprayed) | | | (Average oocyst output per bird) | | |
| Cavitation | E. maxima | E. tenella | E. acervulina | E. maxima | E. tenella | E. acervulina |
| Oocysts | 7/15 (47%) | 5/15 (40%) | 15/15 (100%) | 8.42 × 10⁴ | 5.09 × 10³ | 8.10 × 10⁴ |
| 1500 psi | 11/15 (73%) | 10/15 (67%) | 15/15 (100%) | 8.27 × 10³ | 1.69 × 10⁵ | 4.35 × 10⁴ |
| Improvement | 26% | 27% | 0% | none | 33.2-fold | none |

No oocysts were observed in the intestinal contents of the untreated control birds. The eye drop control for this experiment yielded higher frequency and amplitude of response as compared to the experimental treatment groups. The 1000 psi treatment demonstrated improved frequency of response for E. maxima and E. tenella and improved amplitude of response for all species. The 1500 psi treatment demonstrated improved frequency of response for E. maxima and E. tenella but the no impact on amplitude for E. maxima and E. acervulina however, there was a large increase in the amplitude response for E. tenella.

As expected, the refined hydrodynamic cavitation technique demonstrated by the cell disruption vessel outperformed the less controllable whipped cream dispenser approach. All species with the exception of E. acervulina showed improved infectivity, and in terms of E. tenella oocyst output, both cell disruption vessel pressure parameters showed demonstrable increases.

a. In Vitro Experiments with Additional Vaccines

In previous sections, methods were described for disrupting oocyst membranes to release sporocysts to improve vaccine performance. The initial testing was performed with a 1^{st} broiler chicken coccidia vaccine in both manual and automated processes. Here, the process of disrupting oocyst membranes in a 2^{nd} broiler chicken coccidia vaccine, a 1^{st} and 2^{nd} layer chicken coccidia vaccine and a 1^{st} turkey coccidia vaccine are described.

For manual processing, vaccine was added to glass beads and vigorously shaken. The number of large, medium and small oocysts were counted and compared pre- and post-manual processing. In addition, an automated system was used, in which an aliquot of vaccine was added to a bead mill and samples processed. In this instance a disposable disperser system (IKA ULTRA-TURRAX Tube Drive system) was used, but any similar system would be expected to yield similar results.

For counting purposes, the oocysts included in the tested vaccines were classified as large, medium, and small based on their sizes as outlined in Conway and Mckenzie (Poultry Coccidiosis: Diagnostic and Testing Procedures, 3rd Edition, June 2007, Wiley-Blackwell). Included in the set tested were the following Eimeria species: E. acervulina, E. adenoeids, E. brunetti, E. hagani, E. meleagrimitis E. mivati, E. maxima, E. necatrix, E. praecox, and E. tenella, from various commercial sources. The set included at least one species of both non-attenuated and attenuated (precocious) strains. Each sample was counted three times and the average listed below.

TABLE 22
Oocyst reduction using a 1^{st} layer chicken coccidia vaccine

| | Total Oocysts per | | | Percent reduction compared to | | | |
|---|---|---|---|---|---|---|---|
| 1^{st} Layer Chicken | Sample(Average) | | | starting material | | | |
| Coccidia Vaccine | Large | Medium | Small | Large | Medium | Small | Total |
| Original material | 8.56 × 10¹ | 1.88 × 10² | 4.46 × 10² | — | — | — | — |
| Manual | 2.22 × 10¹ | 5.89 × 10¹ | 1.58 × 10² | 74% | 69% | 65% | 67% |
| Automated | 2.56 × 10¹ | 9.67 × 10¹ | 2.11 × 10² | 70% | 49% | 53% | 54% |

TABLE 23
Oocyst reduction using a 2^{nd} layer chicken coccidia vaccine

| | Total Oocysts per | | | Percent reduction compared to | | | |
|---|---|---|---|---|---|---|---|
| 2^{nd} Layer Chicken | Sample (Average) | | | starting material | | | |
| Coccidia Vaccine | Large | Medium | Small | Large | Medium | Small | Total |
| Original material | 1.86 × 10² | 4.94 × 10¹ | 1.26 × 10³ | — | — | — | — |
| Manual | 3.33 × 10¹ | 1.72 × 10² | 4.10 × 10² | 82% | 65% | 67% | 68% |
| Automated | 6.89 × 10¹ | 2.08 × 10¹ | 5.98 × 10² | 63% | 58% | 52% | 55% |

TABLE 24
Oocyst reduction using a 2^{nd} broiler chicken coccidia vaccine

| | Total Oocysts per | | | Percent reduction compared to | | | |
|---|---|---|---|---|---|---|---|
| 2^{nd} Broiler Chicken | Sample (Average) | | | starting material | | | |
| Coccidia Vaccine | Large | Medium | Small | Large | Medium | Small | Total |
| Original material | 4.22 × 10² | 3.43 × 10² | 1.24 × 10³ | — | — | — | — |
| Manual | 6.22 × 10² | 1.66 × 10² | 4.32 × 10² | 85% | 52% | 65% | 67% |
| Automated | 5.33 × 10¹ | 1.47 × 10¹ | 5.82 × 10² | 87% | 57% | 53% | 61% |

TABLE 25
Oocyst reduction using a 1^{st} turkey coccidia vaccine

| | | Percent reduction |
|---|---|---|
| 1^{st} Turkey | Total Oocysts per | compared to |
| Coccidia | Sample (Average) | starting material |
| Vaccine | Medium* | Medium |
| Original material | 3.11 × 10² | |
| Manual | 1.12 × 10² | 64% |
| Automated | 1.20 × 10¹ | 61% |

*No large or small oocysts present in the vaccine.

Results indicate general susceptibility of oocysts to shearing for a wide variety of vaccines tested, indicating that the systems and processes described have broad applicability.

### Generalized Statements of the Disclosure

The following numbered statements provide a general description of the disclosure and are not intended to limit the appended claims.

Statement 1: A method of vaccinating an animal against Eimeria comprising the steps of: providing a solution of Eimeria oocysts, the oocysts having an outer membrane and containing viable sporocysts therein; disrupting at least some of the Eimeria oocyst outer membranes which results in a modified solution; and delivering the modified solution to an animal.

Statement 2: A method of protecting an animal against an Apicomplexan disorder comprising the steps of: providing a solution of Apicomplexa oocysts, the oocysts having an outer membrane and containing viable sporocysts therein; disrupting at least some of the Apicomplexa oocyst outer membranes which results in a modified solution; and delivering the modified solution to an animal.

Statement 3: The method of any of Statements 1-2, the viable sporocysts are released from the disrupted membrane.

Statement 4: The method of any of Statements 1-3, where the modified solution is delivered to an animal in at the time of disrupting the membranes.

Statement 5: The method of Statement 1-3, where the modified solution is delivered to the animal within 5 days of the disruption which results in the modified solution.

Statement 6: The method of any of Statements 1-5, where the modified solution is delivered by spray.

Statement 7: The method of any of Statements 1-6, where the Eimeria or Apicomplexa oocysts are Eimeria oocysts of single Eimeria species or Apicomplexa oocysts from a single Apicomplexa species.

Statement 8: The method of any of Statements 1-6, where the Eimeria or Apicomplexa oocysts are Eimeria oocysts from an Eimeria vaccine containing two or more Eimeria species or Apicomplexa oocysts from two or more Apicomplexa species.

Statement 9: The method of any of Statements 1-8, wherein the solution of Eimeria or Apicomplexa oocysts is a concentrated vaccine solution.

Statement 10: The method of any of Statements 1-8, wherein the solution of Eimeria or Apicomplexa oocysts is a diluted vaccine solution.

Statement 11: A system for disrupting an outer membrane of Eimeria oocysts, and delivering the resulting solution to an animal in real-time, the system comprising: a vessel containing Eimeria oocysts in a solution, the oocysts having an outer membrane and containing viable sporocysts therein; an oocyst processing chamber where the outer membrane of at least some of the Eimeria oocysts are disrupted which results in a modified solution; and a delivery outlet, whereby the modified solution is moved from the vessel through the processing chamber to the delivery outlet where the modified solution is delivered to an animal.

Statement 12: A system for disrupting an outer membrane of Apicomplexa oocysts, and delivering the resulting solution to an animal in real-time, the system comprising: a vessel containing Apicomplexa oocysts in a solution, the oocysts having an outer membrane and containing viable sporocysts therein; an oocyst processing chamber where the outer membrane of at least some of the Apicomplexa oocysts are disrupted which results in a modified solution; and a delivery outlet, whereby the modified solution is moved from the vessel through the processing chamber to the delivery outlet where the modified solution is delivered to an animal.

Statement 13: The system of Statements 11-12, where the viable sporocysts are released from the disrupted membrane.

Statement 14: The system of any of Statements 11-13, wherein the oocyst processing chamber is at least one of a group consisting of: a high pressure homogenizer, a rotor stator mixer, a chamber vessel containing hard beads and an agitator attached thereto, a pair of vibrating plates, an ultrasonicator, hydrodynamic cavitation device, high pressure sprayer, or a combination thereof.

Statement 15: The system of any of Statements 11-14, wherein the homogenizer provides a pressure of about 3000 psi.

Statement 16: The system of any of Statements 11-15, wherein the number of Eimeria or Apicomplexa oocysts ruptured is between about 5 and 50% for oocysts smaller than 20 microns on their longest dimension, between about 15 and 75% for oocysts ranging in size between 20 microns and 30 microns on their longest dimension, and between about 25 and 90% for oocysts larger than 30 microns on their longest dimension.

Statement 17: The system of any of Statements 11-16, wherein the solution containing the Eimeria or Apicomplexa oocysts includes at least one proteolytic enzyme.

Statement 18: The system of Statement 17, where the proteolytic enzyme is trypsin, chymotrypsin or a mixture thereof.

Statement 19: The system of any of Statements 11-18, wherein the solution containing the Eimeria or Apicomplexa oocysts is a concentrated vaccine solution.

Statement 20: The system of any of Statements 11-18, wherein the solution containing the Eimeria or Apicomplexa oocysts further comprises an aqueous diluent which comprising buffer salts; sugars; proteins or protein hydrolysates; dyes; or thickeners.

Statement 21: A method of disrupting oocyst membranes at the time of delivery to an animal, the method comprising the steps of: providing a vessel for containing a volume of Eimeria oocysts in solution, the oocysts having an outer membrane and containing viable sporocysts therein; providing a system for disrupting the outer membrane of the oocyst; providing a delivery device; moving the solution from the first vessel into the system; passing the solution through the processing chamber, whereby at least some of the Eimeria oocyst membranes are disrupted which results in a modified solution; and moving the modified solution from the system to the delivery device where the modified solution is delivered to an animal.

Statement 22: A method of disrupting oocyst membranes at the time of delivery to an animal, the method comprising the steps of: providing a vessel for containing a volume of Apicomplexa oocysts in solution, the oocysts having an outer membrane and containing viable sporocysts therein; providing a system for disrupting the outer membrane of the oocyst; providing a delivery device; moving the solution from the first vessel into the system; passing the solution through the processing chamber, whereby at least some of the Apicomplexa oocyst membranes are disrupted which results in a modified solution; and moving the modified solution from the system to the delivery device where the modified solution is delivered to an animal.

Statement 23: The method of any of Statements 21-22, wherein the system comprises at least one from the group consisting of a high-pressure homogenizer, an ultrasonicator, a rotor stator mixer, a vessel containing hard beads therein and an agitator attached thereto, a pair of vibrating plates, a hydrodynamic cavitation device, a high pressure sprayer, or a combination thereof.

Statement 24: The method of any of Statements 21-23, wherein the homogenizer provides a pressure of greater than about 3000 psi.

Statement 25: The method of any of Statements 21-24, wherein the number of oocysts ruptured is at least about 5% for oocysts smaller than 20 microns on their longest dimension, at least about 15% for oocysts ranging in size between 20 microns and 30 microns on their longest dimension, and at least about 25% for oocysts larger than 30 microns on their longest dimension.

Statement 26: The method of any of Statements 21-25, wherein the solution of Eimeria oocysts or Apicomplexa is a concentrated vaccine solution.

### ADDITIONAL DISCLOSURE

The present disclosure provides additional embodiments, systems, methods, components, configurations, and applications relating to vibrating-rod cell disruption technology.

Various methods and systems are currently used for preparing pharmaceutical substances from lysed cells, but the present invention should not be limited to this specific application and may be applicable to other applications. Many such methods, however, present drawbacks that may be reduced or avoided by the cell disruption technology described herein. In certain embodiments, a cell suspension is pumped through a disruptor module comprising a tube or chamber containing rods that are vibrated at a frequency effective to disrupt cellular enclosures and release intracellular contents into the surrounding solution. Methods and systems using vibrating-rod technology may provide distinct advantages relative to conventional cell lysis technologies.

### Comparison with Other Cell Disruption Technologies

### Detergent Lysis

Detergent-mediated lysis can denature components of interest, including native or recombinant proteins, particularly with prolonged exposure. Selection of an appropriate detergent may require extensive experimentation. The use of detergent also increases process cost, especially at larger scales. Once added, the detergent remains active unless the medium is exchanged, and detergent residues may render the preparation unsuitable for direct administration to an animal subject without further processing. In general, embodiments using vibrating rods do not use detergent for disruption, and the suspended material is exposed to disruptive forces only during passage through the disruption chamber, which may last only a fraction of a second.

### Enzymatic Lysis

Enzymatic digestion used to cause cell lysis may damage components of interest, including native or recombinant proteins, especially when exposure time is prolonged. Selection of a suitable enzyme or enzyme combination may also require extensive experimentation. Once an enzyme is introduced, enzymatic activity continues unless the medium is exchanged or the enzyme is inactivated. Enzymatic lysis may also leave residues that are harmful or otherwise unsuitable for direct delivery to an animal subject without further processing. In general, in embodiments using vibrating rods, no enzyme is required, and the suspension is subjected to disruptive forces only during transit through the disruption chamber.

### Bead Mill Homogenization

Bead mill processing may subject the entire batch to prolonged shear forces and substantial heat generation. By contrast, the vibrating-rod continuous-flow disruption system described herein generates only minimal heat and can be adapted for a wide range of sample volumes. Unlike batch methods such as bead mills, which expose the full sample to shear for the duration of processing, the present vibrating-rod continuous-flow disruption system subjects material to disruptive forces only during passage through the disruption chamber.

### Ultrasonic Homogenization

Ultrasonic homogenization requires high power input and generates substantial heat. In general, embodiments using vibrating rods do not have high power requirements and do not generate a significant amount of heat. Furthermore, the vibrating-rod continuous-flow disruption system is easily scalable through the addition of disruption segments and, if desired, the implementation of multiple passes through the system.

### Pressure Homogenization

Pressure-based homogenization relies on high operating pressures. In contrast, the vibrating-rod continuous-flow disruption system provides high flow rates without requiring high pressure.

### Temperature-Based Treatments

Heat treatment used to lyse cells may denature proteins and alter three-dimensional structure or conformation, thereby affecting protein function. In general, embodiments using vibrating rods do not generate elevated temperatures, thereby eliminating the risk of heat-induced protein denaturation.

### General System Description

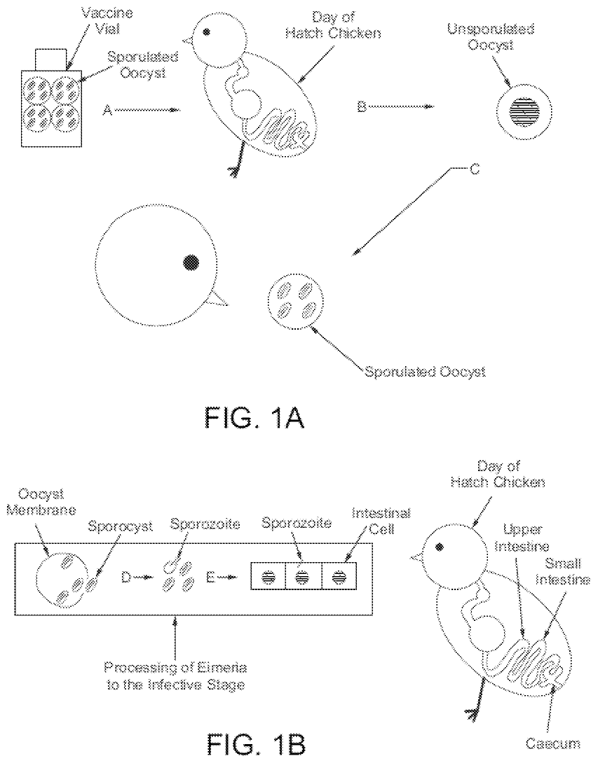

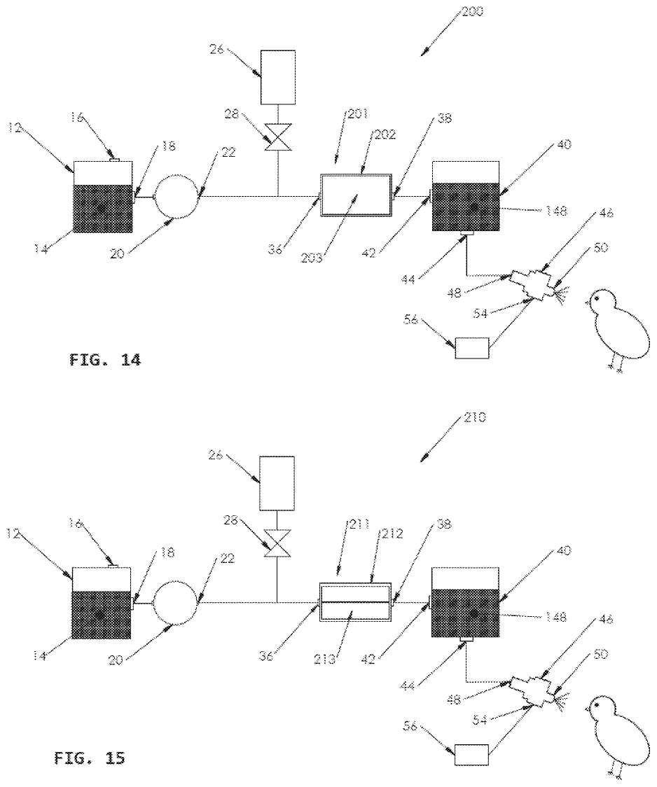

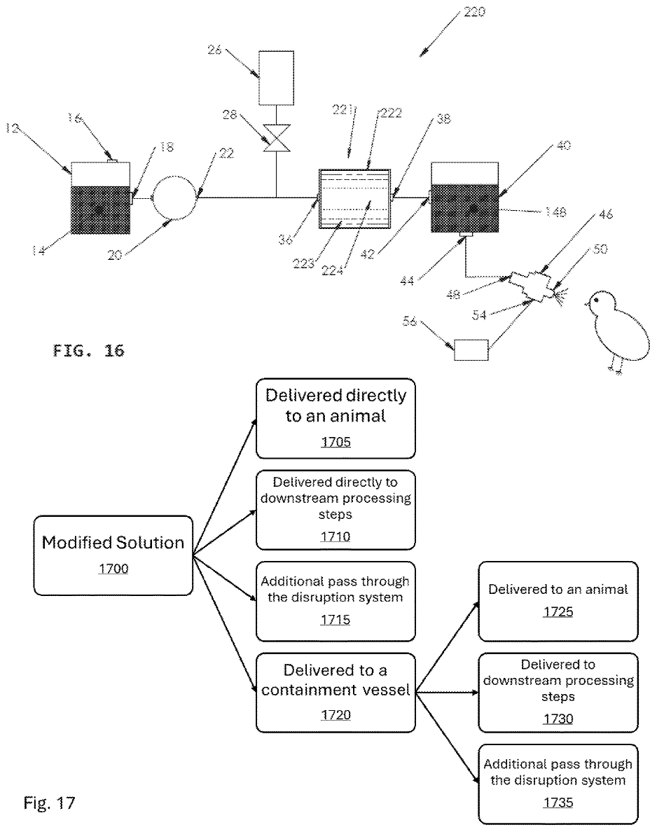

Referring now to various embodiments of the present disclosure, the present disclosure provides improvements over prior system. Methods and systems are provided for disrupting cells and recovering intracellular contents, similar to the embodiments discussed in FIGS. 1-9. In this regard, according to certain embodiments, a cell suspension is pumped through a disruptor module comprising a tube or chamber, but the chambers according to the embodiments of FIGS. 10-17 contain rods that are vibrated at a frequency effective to disrupt cellular enclosures and release cellular contents into solution. This system may be referred to herein as “the disruption system.”

For applications involving delivery of disrupted oocyst vaccines to day-old chicks, the disruption system may form one component of a larger device installed in a facility, such as a hatchery. In certain embodiments, this larger device may receive chicks from an eggshell separator, divide the chicks into lanes, align the chicks in single file, and space the chicks such that a gap exists between adjacent chicks. A vision system and algorithm may be used to identify a target feature, such as the eye of a bird, to enable targeted delivery of disrupted vaccine through a nozzle or an orifice plate.

The larger vaccination device may be configured to meet the daily capacity requirements of large, medium, and small broiler hatcheries or layer hatcheries. In certain embodiments, capacities for broiler operations may range from about 300,000 to about 1,000,000 chicks per day for large operations, about 100,000 to about 250,000 chicks per day for medium operations, and about 5,000 to about 50,000 chicks per day for small operations. In certain embodiments, capacities for layer operations may range from about 100,000 to about 200,000 chicks per day for large operations, about 20,000 to about 50,000 chicks per day for medium operations, and about 500 to about 5,000 chicks per day for small operations. Such systems may be configured as modular components having varying lane structures that may be combined to match hatchery throughput requirements. In certain embodiments, vaccination processes at hatcheries may be completed on the day of hatch within a period of about 8 hours to about 10 hours. Thus, throughput may range from about 5,000 chicks per hour for a small layer operation to more than about 100,000 chicks per hour for a large broiler operation. In certain embodiments, the disruption system may receive an intact sporulated oocyst vaccine and disrupt the oocysts to release intact, viable sporocysts for delivery within the larger vaccination device.

In certain embodiments, cells may be disrupted in the medium in which they are prepared, without requiring the addition of chemicals such as detergents or enzymes. Alternatively, prior to disruption, the cells may be transferred into a different medium, typically a less chemically complex medium such as water or buffer, by techniques including tangential flow filtration, centrifugation, and resuspension.

The present method, according to one or more embodiments, is a flow-through process rather than a batch process. Total exposure of cells and released contents to the disruptive forces may therefore be brief and substantially consistent throughout the preparation. Material disrupted at the beginning of the process is not necessarily subjected to continued disruptive forces for the full duration of the run, in contrast to many batch methods. Instead, material may be exposed primarily during passage through the disruption module, which may occur in only a fraction of a second. Multiple passes may be used, if desired, to increase disruption efficiency. Although not generally required, compounds such as detergents, enzymes, or buffers may optionally be included in the solution.

### Materials and Surface Treatments

In certain embodiments, vibration system components, including housings, cylinders, plates, rods, and outlet components such as nozzles or orifice plates (see, e.g., FIG. 10), may be constructed from metals including 304 stainless steel, 316 stainless steel, titanium, or other suitable materials. Components may be cleaned using commercial detergent formulations to remove oil or grease residue and may be passivated, for example, with citric acid or nitric acid solutions, to remove rust if needed.

Components may also be treated with coatings to improve longevity or modify surface properties. In certain embodiments, coating processes such as chemical vapor deposition may be used to provide hydrophilic or hydrophobic surfaces. Chemical vapor deposition may provide uniform, highly conformal coatings of high purity and strong adhesion, including on parts with complex geometries. Other surface-treatment processes may also be used, including physical vapor deposition, atomic layer deposition, and plasma-assisted variants of chemical vapor deposition. Such treatments may provide one or more desirable properties, including precision thickness, adhesion, density, wear resistance, and corrosion resistance.

### Modular Configurations and Scale-Up

The vibrating-rod disruption systems according to various embodiments of the present disclosure may be constructed as modules having varying functionality, including different rod diameters, capacities, or vibrational ranges. These modules may be configured for high-capacity, intermediate-capacity, or low-capacity operation. In certain embodiments, modules may be used in sets designed for particular products and may be sanitized or sterilized using clean-in-place, steam-in-place, or other suitable protocols.

The process may be scaled by increasing the size of disruption modules and/or by increasing the number of modules. Modules may be arranged in series, in parallel, or in combinations thereof. Process efficiency may also be improved by passing the cell suspension through the disruption system multiple times.

In certain embodiments, the method may operate at relatively high flow rates without incurring high backpressure. High-pressure chambers are not required in certain implementations. The method may further operate without generating excessive or damaging heat or sound.

### Monitoring and Filtration

The method may include one or more particle size detectors to evaluate the size of the particles of the sporocyst or any other items in the system so as to determine the extent of processing. Such detectors may be positioned downstream of the disruption system or both upstream and downstream of the disruption system. Alternatively, samples may be collected before disruption, after disruption, or both before and after disruption and may be examined microscopically, among other techniques, to evaluate disruption efficiency.

The system may also include perforated-disc filters or mesh-screen filters within the vibrating section to retain rod components and/or remove oversized particles. Vibration may assist in reducing screen blockage and maintaining flow, even if some debris accumulates.

### Variables Affecting System Performance

Numerous aspects and component features may affect system performance and may be varied to improve function in particular applications, including, without limitation, (1) the length of the disruption chamber, (2) the geometry of the disruption chamber, (3) the diameter of the disruption chamber, (4) the total volume of the disruption chamber, (5) the size of the rods used in the disruption chamber, (6) the shape of the rods used in the disruption chamber, (7) the diameter of the rods, (8) the number of rods used in the disruption chamber, (9) mixtures of different rod types, (10) the total interstitial space within the disruption unit, defined as the volume of the chamber less the volume occupied by the rods, (11) packing density, including the ratio of rod volume to interstitial volume, (12) the materials of construction of chamber components and rods, (13) surface treatments, including hydrophilic or hydrophobic coatings on chamber walls, rods, nozzles, or orifice plates, (14) the design and materials of construction of retention screens used to contain rods within the chamber, (15) the flow rate through the disruption chamber, (16) the chemical composition of the medium in which cells are suspended during passage through the chamber, (17) vibration frequency, (18) variation in vibration frequency during operation, (19) vibration amplitude, including amplitude controlled by weights in the vibratory mechanism, and/or (20) the temperature of the chamber and/or of the suspension medium. This list is not intended to be exhaustive and thus, the present invention should not be limited to just this list of features and may include other features in combination with, instead of or in addition to the above listed feature possibilities. In this regard, other aspects and features may also contribute to system performance. Multiple configurations may also be used in series to achieve a desired result. For example, a chamber containing relatively large rods may be used to disrupt larger enclosures, followed by one or more chambers containing smaller rods to achieve finer disruption. Optimization of these and other parameters for a given application may be determined empirically.

### Retention Screens

Retention screens may be provided in various forms, including wire-mesh screens and perforated-disc screens. The opening size may be selected based on rod size so that the rods are efficiently retained. Opening size may also be selected to exclude or retain particles above a desired diameter, thereby providing a filtering function. Vibration may assist flow through the retention screens and may reduce blockage by retained particles.

### Packing Density Considerations

Spherical beads are commonly used for cell disruption in bead mills, but the packing characteristics of spheres may impose certain limitations. For identical spheres in three dimensions, the densest theoretical packings are hexagonal close packing and face-centered cubic packing, each having a packing density of approximately 74%. In practice, however, random shaking of spherical beads does not produce perfect geometric packing and instead yields irregular random close packing, which may be about 64%. Variation in bead diameter may further reduce efficiency.

By contrast, the packing density of rods parallel to each other in a cylindrical housing may be approximated from the two-dimensional packing density of circles in a plane. The densest packing of identical circles in a plane is hexagonal packing, which achieves a theoretical density of about 90.7%. In certain embodiments, vibrating rods in a cylindrical housing may be forced toward a closely packed configuration at each peak and trough of the vibration amplitude, potentially producing densities closer to the theoretical two-dimensional close-packed value and appreciably greater than the density typically achieved with randomly packed beads. As described herein, vibrating rods have been shown to be useful in disrupting cells such as Eimeria oocysts having diameters in the range of about 16 μm to about 30 μm while allowing recovery of intact internal sporocysts.

Additional embodiments employing closely matched surfaces, such as vibrating plates, a single vibrating rod within a cylindrical housing, or concentric cylinders with a central rod, may provide effective packing densities greater than 90%, limited in part by surface smoothness and manufacturing tolerances. Such embodiments may provide improved efficiency for disrupting cells having diameters below about 16 μm. As packing density increases, however, fluid flow may become restricted, and optimization of remaining interstitial space may be desirable to maintain suitable flow.

The foregoing aspects, individually and in combination, provide methods and systems for disrupting cellular structures in a manner distinct from conventional cell lysis technologies. Production of subcellular pharmaceutical substances from living cells is a substantial and growing field that may benefit from the methods and systems described herein, as may recipients of medicines produced using such methods and systems.

### Vaccine Preparation and Broader Applications

Preparation of an Eimeria vaccine at a hatchery may involve dilution of a live, sporulated oocyst-based vaccine to yield a 1× dose in a prescribed volume. The oocyst vaccine may comprise wild-type species, attenuated species, or a combination thereof. In certain embodiments, the attenuated species may comprise one or more selected precocious lines. Genetically modified species may also be used. A concentrated commercial oocyst-based vaccine may be prepared at a prescribed dilution and delivered to a receiving tank, which may serve as a first vessel for containment of unprocessed cells.

In certain embodiments, the larger vaccine delivery device may be deployed in a commercial hatchery and may receive day-of-hatch chicks from an eggshell separator device on a moving conveyor. The device may partition the birds into multiple lanes, singulate the birds, generate gaps between individual chickens, and present the gapped chickens to a vision system configured to collect images, analyze bird position, identify a target such as the eye, and actuate a delivery system to apply a measured dose of disrupted vaccine. The disruption system may include a chamber or housing containing vibrating rods and one or more delivery components, such as a nozzle or an orifice plate containing microchannels configured to deliver the vaccine by spray or stream. In certain embodiments, disrupted vaccine may be held temporarily in a containment vessel that is stirred, agitated, or recirculated to maintain a well-mixed composition of the solution and suspended components.

The vaccine dose delivered to a single chicken may have a volume in the range of about 1 μL to about 14 μL, about 10 μL to about 20 μL, about 15 μL to about 30 μL, about 25 μL to about 50 μL, or about 35 μL to about 100 μL, for example. A system for stirring, agitating, or recirculating the modified solution in the containment vessel may be configured to ensure a sufficiently similar distribution of infective parasite life stages within the administered dose so as to achieve similar infectivity and similar development of immunity among vaccinated subjects. The disrupted vaccine may be delivered individually to in-motion avian subjects in a high-throughput manner using a nozzle or system of nozzles, or an orifice plate or system of orifice plates, via actuated targeting systems. In certain embodiments, each lane of the vaccination device may vaccinate about 8,000 to about 12,000 conveyed birds per hour or more while operating in an in-motion, high-throughput manner.

In broader applications, the cell disruption system using vibrating rods may, in various embodiments, be applied to pharmaceutically relevant processes for production of subcellular substances from cells, tissues, organoids, or similar structures, thereby yielding recoverable intracellular structures, DNA, RNA, native proteins, and recombinant proteins from a variety of cell sources, including but not limited to Apicomplexan parasite cells, bacterial cells, yeast cells, fungal cells, plant cells, insect cells, mammalian cells, and other cell lines, and at various scales, such as laboratory scale, research scale, and manufacturing scale. Methods and systems using vibrating rods may also be applicable in bulk viral vaccine production systems, such as bioreactors, to recover viruses from cells. In certain embodiments, the system provides efficient cell disruption in continuous-flow mode generally without the need for addition of chemicals such as detergents or enzymes, and without the need for costly high-pressure systems, heat-generating mechanical systems, or ultrasonic probe systems.

Methods and systems using vibrating rods for disruption of cells may operate in a continuous-flow manner, thereby overcoming disadvantages of batch disruption methods, including overprocessing and heat generation. The flow-through system may ensure high recovery of processed sample materials. The vibrating system may efficiently disrupt both dilute and concentrated suspensions. The vibrating-rod disruption system may be constructed in modules with varied capacities, and the modules may be combined in series or parallel configurations to yield higher capacity and higher efficiency. The vibrating-rod disruption system may further be constructed to function in conjunction with various pumps, valves, and containment vessels such that multiple passes of the cell suspension through the disruption system may accomplish more complete cell disruption than a single pass. Modules may also be designed for use in series such that successively smaller cellular components are disrupted.

In certain embodiments, the cell disruption system may include a first vessel containing unbroken cells in suspension, a cell processing chamber containing rods and connected to a source of vibration, and a downstream receiving vessel, similar to the systems discussed above with regard to FIGS. 1-9. Also similar to FIGS. 1-9, Fluid may be moved through the disruption chamber by a pump, gravity feed, pressure differential, or another liquid-transfer mechanism. The cells may be processed in the medium in which they were produced or may first be concentrated, washed, or resuspended in water, buffer, or another formulation before passage through the disruption chamber.

In certain embodiments, the disclosed systems may be used to disrupt cell enclosures, including cell walls and/or membranes, and to direct the resulting modified suspension either to a containment vessel, to downstream processing, or to delivery to an animal. In certain embodiments, the intact cells may comprise Eimeria oocysts, and the released subcellular structures may include viable Eimeria sporocysts for use as a coccidiosis vaccine for chickens.

The methods and systems described herein may be used to replace current cell disruption methods and systems such as chemical lysis, enzymatic lysis, microfluidic shearing, or bead mill disruption. The methods and systems may be applied to large-scale production, intermediate-scale production, or small-scale research production. The methods and systems may also be used in diagnostic applications.

The methods and systems may be applicable to disruption of a variety of cell types used to produce pharmaceutical substances, including, but not limited to, cells derived from archaea and fungi, as well as cell lines obtained from animals including insects, fish, reptiles, mammals, birds, and humans. Such cell types may include mammalian, but not limited to, cell lines such as Chinese hamster ovary (CHO) cells, human embryonic kidney (HEK293) cells, HeLa cells, mouse myeloma cells (NS0 and Sp2/0), baby hamster kidney (BHK) cells, Madin-Darby canine kidney (MDCK) cells, Vero cells, and chick embryo fibroblasts (CEFs).

Microbial cells such as bacteria and yeast may also be lysed using the described methods and systems. Bacterial cells may include, but are not limited to, Escherichia coli, Bacillus species such as Bacillus subtilis, Corynebacterium glutamicum, and Streptomyces species. Yeast cells may include, but are not limited to, Saccharomyces cerevisiae, Komagataella phaffii (formerly Pichia pastoris), Hansenula polymorpha, Kluyveromyces lactis, Yarrowia lipolytica, and Schizosaccharomyces pombe.

Plant cell lines may also be used and may include, but are not limited to, tobacco BY-2 cells, rice cells, carrot cells, alfalfa cells, tomato cells, and soybean cells. Insect cell lines may also be used and may include, but are not limited to, those derived from the fall armyworm, including Sf9 and Sf21 lines, from the fruit fly, including S2 cells, from the silkworm Bombyx mori, or from other lepidopteran species.

While the above-mentioned cells and cell lines cover many applications, numerous other cell lines may also be used for production of subcellular substances, and recovery of such substances from those cell lines may benefit from employment of the present disruption technology. Additional applications may include homogenization and disruption of tissues or organoid structures for use in personalized medicine, pharmaceutical development, or diagnostic methodologies.

Other applications may include processing harvested grains or cultured microbes such as yeast to derive components for use in human food products or animal feed. Further applications may include recovery of fertilizer components from waste materials, including phosphorus-containing or nitrogen-containing compounds. Waste materials or waste streams may include, for example, spent brewer's yeast, algal biomass residue, poultry litter, or municipal sewage.

Some plants, such as the fern Blechnum orientale, have been found to absorb rare earth elements from soil and, in a process referred to as phytomineralization, hyperaccumulate those elements within plant tissues as monazite crystals. Extraction and recovery of mineral crystals from plant tissues is referred to as phytomining. A similar process can be used with fungi, a process known as mycomining. Systems and methods of disrupting plant or fungal cells using vibrating-rod technology may therefore be applicable to phytomining or mycomining processes for recovery of rare earth elements from plant or fungal biomass.

### Additional Embodiments of Vibrating-Rod Processing Systems

### Ninth Embodiment—Vibrating Rods

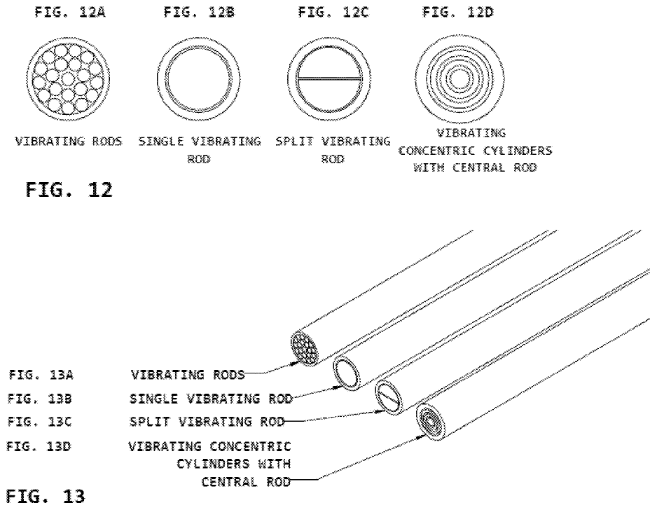

A ninth embodiment 190 is shown in FIG. 11 and is similar to the first embodiment 10, second embodiment 60, third embodiment 80, and fourth embodiment 120 described above, except for processing system 191. The ninth embodiment 190 includes a first reservoir 12 and pump 20. The ninth embodiment 190 further includes a ninth processing system 191, within containment 192, made up of a tube filled with smaller-diameter cylindrical rods 193. The rods 193 are packed into the tube such that most of the space within the tube is occupied by the rods, as shown in FIGS. 12A and 13A, while leaving sufficient space for the rods to vibrate. The tube containing the rods is connected to a vibration mechanism (not shown). The vibration mechanism causes the tube 192 to vibrate and the rods 193 to vibrate within the tube. The rods 193 may have varying diameters as needed to disrupt cell enclosures, including cell walls and/or cell membranes, during passage of the cells within the tube.

In use, cell suspension 14 is moved into the ninth processing system 191. A flow of suspension 14 is directed into the tube 192 so that the suspension 14 is allows to flow or travel between the rods 193. When the suspension 14 flows or travels between the rods 193, the vibration mechanism is activated, causing the rods 193 to vibrate between and/or against each other. As suspension 14 moves between the rods 193 while the vibration mechanism is activated, at least some of the membranes and/or walls of the cells in suspension 14 are disrupted as the cells pass between the rods, thereby creating a modified suspension 148. The modified suspension 148 may be delivered to a holding container, such as reservoir 40. The modified suspension may then be delivered to a delivery device 46 for delivery to a subject or to other system components for additional processing, including passage back through the system. Alternatively, the modified suspension 148 may be delivered directly to the delivery device 46 or to other system components for additional processing, including passage back through the system. In this regard, the suspension 14 enters one end of the tube 192 with the membranes in tact, travels between the rods 193 while the rods 193 vibrate, and then exit the other end of the tube 192 with the membranes broken, thereby resulting the oocyst being released out of the other end of the tube 192. This allows for efficient releasing of the oocyst from the membranes.

FIG. 12 shows cross-sectional views of processing systems 191, 201, 211, and 221, as shown in FIGS. 12A, 12B, 12C, and 12D, respectively. The embodiment of FIG. 12A is discussed above, while FIGS. 12B, 12C, and 12D are discussed below as the tenth, eleventh and twelfth embodiments, respectively. FIG. 13 shows isometric views of processing systems 191, 201, 211, and 221, as shown in FIGS. 13A, 13B, 13C, and 13D, respectively.

### Tenth Embodiment—A Single Vibrating Rod

A tenth embodiment 200 is shown in FIG. 14 and FIGS. 12B and 13B and is similar to the ninth embodiment 190 described above, except for processing system 201. The tenth embodiment 200 includes a first reservoir 12 and pump 20. The tenth embodiment 200 further includes a tenth processing system 201, within containment 202, made up of a tube containing a single smaller-diameter cylindrical rod 203. The rod 203 fits into the tube such that most of the space within the tube is occupied by the rod, as shown in FIGS. 12B and 13B, while leaving sufficient space for the rod to vibrate. The tube containing the rod is connected to a vibration mechanism (not shown). The vibration mechanism causes the tube 202 to vibrate and the rod 203 to vibrate within the tube. The rod 203 may have varying diameters as needed to disrupt cell enclosures, including walls and/or membranes, during passage of the cells within the tube.

In use, cell suspension 14 is moved into the tenth processing system 201. A flow of suspension 14 is directed into the tube and around the rod 203. The vibration mechanism is activated, causing the rod 203 to vibrate against the interior of the tube. As suspension 14 moves between the interior wall of the tube 202 and the rod 203, at least some of the cell enclosures of the cells in suspension 14 are disrupted as the cells pass between the rod and the tube, thereby creating a modified suspension 148. In this regard, the suspension 14 enters one end of the tube 202 with the membranes in tact, travels between an outer diameter of the single rod 203 and an inner diameter of the tube 202 while the rod 203 vibrates, and then exits the other end of the tube 202 with the membranes broken, thereby resulting the oocyst being released out of the other end of the tube 202. This allows for efficient releasing of the oocyst from the membranes.

After the oocyst is released, the modified suspension 148 may be delivered to a holding container, such as reservoir 40. The modified suspension may then be delivered to a delivery device 46 for delivery to a subject or to other system components for additional processing, including passage back through the system. Alternatively, the modified suspension 148 may be delivered directly to the delivery device 46 or to other system components for additional processing, including passage back through the system.

### Eleventh Embodiment—Split Vibrating Rod

An eleventh embodiment 210 is shown in FIGS. 15, 12C and 13C and is similar to the ninth embodiment 190 and tenth embodiment 200 described above, except for processing system 211 is different. The eleventh embodiment 210 includes a first reservoir 12 and pump 20. The eleventh embodiment 210 further includes an eleventh processing system 211, within containment 212, made up of a tube containing a single cylindrical rod split lengthwise through the middle to form rod-halves 213. In this regard, each of the two rod-halves 213 have a semi-circular cross-section and have a space between each other for the suspension to travel therebetween. In this regard, openings through which fluid may pass include the gap between the semi-cylindrical halves of the rod and the inner straight passage through the middle of the rod. The rod-halves are packed into the tube such that most of the space within the tube is occupied by the rod-halves, as shown in FIGS. 12C and 13C, while leaving sufficient space for the rod-halves to vibrate. The tube containing the rod-halves is connected to a vibration mechanism (not shown) configured to vibrate the rod-halves. The vibration mechanism causes the tube 212 to vibrate and/or the rod-halves 213 to vibrate within the tube. The containment cylinder and the rod-halves 213 may have varying diameters as needed to disrupt cell enclosures, including walls and/or membranes, during passage of the cells through the system.

In use, cell suspension 14 is moved into the eleventh processing system 211. A flow of suspension 14 is directed into the tube and between the rod-halves 213. The vibration mechanism is activated, causing the rod-halves 213 to vibrate against each other and against the cylindrical walls of the containment tube. As suspension 14 moves through the central passage between the rod-halves and along the exterior curved surfaces of the rod-halves 213, at least some of the membranes and/or walls of the cells in suspension 14 are disrupted, thereby creating a modified suspension 148. The modified suspension 148 may be delivered to a holding container, such as reservoir 40. The modified suspension may then be delivered to a delivery device 46 for delivery to a subject or to other system components for additional processing, including passage back through the system. Alternatively, the modified suspension 148 may be delivered directly to the delivery device 46 or to other system components for additional processing, including passage back through the system. In this regard, the suspension 14 enters one end of the tube 212 with the membranes in tact, travels between an outer diameter of the rod-halves 213 and an inner diameter of the tube 212 and/or in between the two rod-halves 213 while the rod-halves 213 vibrate, and then exits the other end of the tube 212 with the membranes broken, thereby resulting the oocyst being released out of the other end of the tube 212. This allows for efficient releasing of the oocyst from the membranes.

### Twelfth Embodiment—Concentric Cylinders with Central Rod

A twelfth embodiment 220 is shown in FIGS. 16, 12D, and 13D and is similar to the ninth embodiment 190, tenth embodiment 200, and eleventh embodiment 210 described above, except for processing system 221 being different. The twelfth embodiment 220 includes a first reservoir 12 and pump 20. The twelfth embodiment 220 further includes a twelfth processing system 221, within containment 222, made up of a tube containing a set of concentric cylinders 223 and a solid central rod 224. The openings through which fluid may pass include the gaps between adjacent cylinders and the gap between the smallest-diameter cylinder and the central rod.

In this regard, the rods are effectively hollow so that the fluid can flow between the outer diameter of one cylinder and an inner diameter of the adjacent larger cylinder for all of the cylinders in the system. The concentric cylinders and the central rod are packed into the tube such that most of the space within the tube is occupied by the cylinders and the rod, as shown in FIGS. 12D and 13D, while leaving sufficient space for the elements to vibrate. The tube containing the cylinders and rod is connected to a vibration mechanism (not shown). The vibration mechanism causes the tube 222 to vibrate and the elements to vibrate within the tube. The containment cylinder and the elements 223 and 224 may have varying diameters as needed to disrupt cell enclosures, including walls and/or membranes, during passage of the cells through the system.

In use, cell suspension 14 is moved into the twelfth processing system 221. A flow of suspension 14 is directed into the tube and between the cylindrical elements and the rod 223, 224. The vibration mechanism is activated, causing the elements to vibrate against each other and against the cylindrical walls of the containment tube. As suspension 14 moves between the cylindrical sections and the exterior curved surfaces of the rod 223, 224, at least some of the membranes and/or walls of the cells in suspension 14 are disrupted, thereby creating a modified suspension 148. The modified suspension 148 may be delivered to a holding container, such as reservoir 40. The modified suspension may then be delivered to a delivery device 46 for delivery to a subject or to other system components for additional processing, including passage back through the system. Alternatively, the modified suspension 148 may be delivered directly to the delivery device 46 or to other system components for additional processing, including passage back through the system. In this regard, the suspension 14 enters one end of the tube 222 with the membranes in tact, travels between an outer diameter of the largest rod of the rods 223 and an inner diameter of the tube 222 and/or in between adjacent rods 223 while the rods 223 vibrate, and then exits the other end of the tube 222 with the membranes broken, thereby resulting the oocyst being released out of the other end of the tube 222. This allows for efficient releasing of the oocyst from the membranes.

### Disposition of Modified Suspension

As shown in FIG. 17, after the cells have traversed and exited the housing and vibrating components of the processing system, the modified solution 1700 may be held at the current level of processing, recirculated to an upstream supply tank to provide a continuous disruption process, or moved to various downstream operations. In vaccination applications, material exiting the disruptor may be directly and immediately sent to a downstream device or module configured for a particular use, for example a vaccine delivery device for direct administration to a subject. Thus, the modified solution 1700 may be delivered directly to an animal 1705 in real time (e.g., within 60 seconds, within 1-60 minutes, within 1-24 hours, within 1-2 days, etc.), or it may be delivered to a containment vessel 1720 and then delivered to an animal 1725 via a nozzle or orifice plate containing a set of microchannels through which the vaccine exits.

In biopharmaceutical production applications, the processed solution may be directed to downstream processing operations including filtration, tangential flow filtration, centrifugation, or other separation or purification steps. The processed solution may alternatively be directed to a containment vessel and then to one or more downstream operations. For example, the modified solution 1700 may be delivered for additional passage through disruption system 1715, or it may be delivered to a containment vessel 1720 and then to disruption system 1715 one or more times to further complete the disruption process 1735. In some embodiments, the processed solution may be collected in a containment vessel after a first pass through the disruption system and then passed through the system one or more additional times until a desired endpoint is reached. The modified solution 1700 may also be delivered to subsequent downstream processing steps 1710, or to a containment vessel 1720 and then to downstream processing steps 1730. The described dispositions of the modified solution are indicative of the types of paths and processes that may be utilized, but various other paths and processes may be used as needed.

### Development of Initial Vibrating-Rod Disruption System

The first vibrating-rod system that was developed consisted of a single 152.4 mm (6 in.) long, 12.7 mm (½ in.) outer-diameter 304 stainless steel tube packed with 2 mm diameter stainless steel rods in quantities of n=15 to 21. The ends of the stainless steel tubing were hydraulically sealed with compression fittings and attached to polymer tubing having an internal diameter of either 3.175 mm (⅛ in.) or 2.38 mm ( 3/32 in.). The chamber containing the agitation media was attached near each end of the tube to a triangular support structure made of 3D-printed PETG and suspended using small-gauge extension springs. The flexible attachment by the extension springs allowed the assembly to vibrate or oscillate loosely. A vibration motor assembly, consisting of an electric motor with its shaft coupled to an eccentric rotating mass, was affixed to the center of the vibration chamber with a 3D-printed PETG housing and was rotationally stabilized with small-gauge extension springs attached to the base of the apparatus. Upon powering the motor, the vibration chamber oscillated in the plane defined by the two axes orthogonal to the axis of liquid flow through the vibration chamber, thereby causing the agitation media within the chamber to vibrate or oscillate correspondingly. Suspensions, including mixed-species Eimeria oocyst vaccine suspensions, were pumped through the vibration chamber, thereby exposing the vaccine to the vibrations or oscillations of the agitation media upon entering one side of the chamber and exiting the other side into a collection vessel. Liquid flow was controlled by a rotary peristaltic pump connected to tubing on the entry side of the vibration chamber.

Other media were tested in addition to rods, including stainless steel beads having diameters of 3 mm, 3.5 mm, or 4 mm; glass beads having diameters of 3 mm or 4 mm; and concentric stainless steel tubing of 14 mm outer diameter (OD)/11 mm inner diameter (ID), 10 mm OD/7 mm ID, and 6 mm OD/4 mm ID, with a 3 mm diameter central rod.

A series of experiments were run to determine the impact of flow rate, vibration amplitude and frequency, as well as agitation media composition, size, and count. These parameters were adjusted, and the disruptive impact on the oocysts was characterized using in vitro assessments of the percent reduction of each Eimeria species in the mixed-species vaccine after exposure to the vibration chamber. Through experimentation and observational feedback, it was determined that packed stainless steel rods provided the most consistent oocyst disruption in the in vitro assessments. The rods also provided the benefits of ease of handling and consistent packing, as compared to bead-style agitation media.

Further developments of the device led to lengthening of the vibration chamber tubes and agitation media, as well as to a symmetrical bilateral tubing configuration that facilitates balance of the vibration motor assembly between two tubes, rather than fixturing the vibration motor assembly to a single tube. The extension springs were replaced with elastomeric rubber, which is less susceptible to fatigue-based failure than the small-gauge wire extension springs. The 3D-printed structural components were also replaced with more robust stainless steel components. A custom eccentric mass, shaft, roller bearing, and brushless motor assembly replaced the off-the-shelf brushed DC vibration motor of the original prototype. The larger-scale version of the device developed during this process facilitated an increase in processing flow rate, as compared to the original version, highlighting the ability of the technology to scale modularly to meet processing requirements. For example, the first tests processed vaccine at 15 mL/min, whereas the current device can process vaccine at more than 20 times the rate of the first tests.

Following the ruggedization efforts for the vibrating-rod disruption module, a combination of in vitro and in vivo studies was run to determine an optimal flow rate for processing the mixed-species vaccine, with the metrics of percent reduction of oocysts, percent of birds positively inoculated with the disrupted vaccine, and oocyst output from inoculated birds guiding the optimization. Disruption performance of the system was assessed at several points across the range of 90 mL/min to 360 mL/min.

### EXAMPLES

Disruption methods were developed, optimized, and tested for consistency over time. Mixed-species oocyst suspensions were prepared and passed through a vibrating-rod system to disrupt the oocysts and release sporocysts. The mixed-species vaccine comprised large oocysts, including E. maxima, about 31 μm along the longest axis; medium oocysts, including E. tenella, about 22 μm along the longest axis; and small oocysts, including E. acervulina, about 18 μm along the longest axis and E. mivati, about 16 μm along the longest axis.

### Example 1—Optimization of Flow Rate

Experiments were conducted to determine the flow rate that optimized both the percent disruption of E. maxima and the recovery of infective sporocysts, as indicated by oocyst output per bird after eyedrop administration of the disrupted vaccine. The disruption assembly consisted of two hollow cylindrical chambers, each composed of 316 stainless steel, in series configuration. Each disruption cylinder had a length of 304.8 mm and a nominal internal diameter of 10.9 mm. The eccentric rotating mass weight was 38 g. The disruption motor frequency was 7,300 rpm. The disruption agitation media were solid cylindrical rods made of 316 stainless steel, 300 mm in length and 2 mm in diameter. Each chamber was packed with 21 rods.

Flow rates were tested in two experiments, Experiment 43 and Experiment 45, in the range of 90 mL/min to 360 mL/min. For each flow rate tested, the percentage of disruption was calculated by microscopically enumerating E. maxima oocysts in the vaccine prior to and after disruption. Also, for each flow rate tested, day-old chickens, in two replicates of 15 birds, were inoculated with disrupted vaccine by eyedrop and grown out to 7 days. Intestinal contents were then collected, and oocysts were enumerated microscopically. The average oocyst output per bird was then calculated. Results are provided in Table 26.

Averaging across the two experiments, optimum recovery of viable released sporocysts, as evidenced by oocyst output on Day 7, was produced by a flow rate of 220 mL/min, which coincided with observed oocyst disruption of approximately 80% and an output of approximately 300,000 large oocysts per bird.

TABLE 26
Flow Rate by Mean % Disruption and Large Oocyst Output
per bird Mean

| Experiment | Experiment | | |
|---|---|---|---|
| 43 Flow | 45 Flow | | Large Oocyst |
| Rate | Rate | % Disruption | Output per bird |
| (mL/min) | (mL/min) | (Mean) | (Mean) |
| 90 | — | 94.74 | 151,792 |
| 110 | — | 92.11 | 178,398 |
| 135 | 135 | 88.59 | 172,741 |
| 175 | 175 | 83.58 | 279,528 |
| 220 | 220 | 80.08 | 314,586 |
| — | 285 | 67.05 | 211,413 |
| — | 360 | 59.47 | 166,037 |

### Example 2—Consistency of Disruption at 30 Total Sporulated Oocysts Per Microliter

A set of disruption studies was run at a concentration of 30 total sporulated oocysts per microliter. Suspensions were periodically prepared and processed using the vibrating-rod disruption system over a period of months.

The disruption assembly consisted of two hollow cylindrical chambers, each composed of 316 stainless steel, in series configuration. Each disruption cylinder had a length of 304.8 mm and a nominal internal diameter of 10.9 mm. The eccentric rotating mass weight was 38 g. The disruption motor frequency was 7,300 rpm. The disruption agitation media were solid cylindrical rods made of 316 stainless steel, 300 mm in length and 2 mm in diameter. Each chamber was packed with 21 rods. Fluid was pumped through the system at a rate of 205 mL/min, achieving a combined residence time of 4.9 seconds for oocysts transiting through the pair of disruption chambers.

For each experiment, samples were taken before and after disruption. Oocysts were enumerated microscopically by size, and the disruption percentage for each size category was calculated. Disruption results for each run in the set of independently prepared and processed suspensions are summarized in Table 27.

TABLE 27
Disruption % at 30 Oocysts/uL
Disruption % at 30 Oocysts/uL

| | % Large Oocysts | % Medium Oocysts | % Small Oocysts |
|---|---|---|---|
| Date | Disruption | Disruption | Disruption |
| 2022-08-04 | 64.95 | 51.22 | 31.48 |
| 2022-08-30 | 69.76 | 52.77 | 23.21 |
| 2022-09-27 | 69.54 | 63.02 | 40.68 |
| 2022-10-11 | 69.82 | 62.93 | 37.93 |
| 2022-10-25 | 64.07 | 51.73 | 33.42 |
| 2022-11-15 | 69.16 | 52.01 | 31.15 |
| 2022-11-29 | 68.45 | 42.91 | 36.64 |
| 2023-01-05 | 63.73 | 35.46 | 29.10 |
| 2023-01-20 | 63.16 | 23.76 | 18.22 |
| 2023-02-17 | 72.59 | 45.04 | 37.07 |
| 2023-03-03 | 67.08 | 38.25 | 39.27 |
| 2023-03-20 | 70.18 | 57.06 | 38.13 |
| 2023-03-30 | 67.77 | 62.99 | 39.57 |
| 2023-04-13 | 66.52 | 57.61 | 38.99 |
| 2023-04-21 | 72.95 | 53.85 | 42.68 |
| 2023-05-11 | 69.80 | 53.20 | 26.50 |
| 2023-05-25 | 66.30 | 60.60 | 26.30 |
| 2023-06-01 | 66.19 | 49.50 | 24.05 |
| 2023-06-20 | 63.59 | 50.12 | 28.22 |
| 2023-07-06 | 65.70 | 45.70 | 40.60 |
| 2023-08-17 | 71.55 | 26.70 | 43.22 |
| 2023-09-28 | 68.30 | 48.90 | 37.60 |
| 2023-10-12 | 67.67 | 54.43 | 33.23 |
| 2023-10-26 | 66.43 | 44.13 | 40.88 |

Summary statistics for the set of samples prepared at 30 total sporulated oocysts per microliter are shown in Table 28. Disruption conditions were tuned to be more efficient for large oocysts. Results for large oocysts were highly repeatable, with a coefficient of variation of 4.10%.

TABLE 28
Disruption % Summary Statistics at 30 Oocysts/uL
Disruption % Summary Statistics at 30 oocysts/uL

| | % Large | % Medium | % Small |
|---|---|---|---|
| | Oocysts | Oocysts | Oocysts |
| Statistic | Disruption | Disruption | Disruption |
| # | 24 | 24 | 24 |
| Observations | | | |
| Mean | 67.72 | 49.33 | 34.09 |
| Disruption % | | | |
| Std Dev. | 2.78 | 10.40 | 6.90 |
| CV % | 4.10 | 21.08 | 20.23 |

### Example 3—Consistency of Disruption at 100 Total Sporulated Oocysts Per Microliter

Another set of disruption studies was run at a concentration of 100 total sporulated oocysts per microliter, or about 3.3-fold more concentrated than the suspensions prepared at 30 total sporulated oocysts per microliter. Suspensions were periodically prepared and processed using the vibrating-rod disruption system over a period of months.

The disruption assembly consisted of two hollow cylindrical chambers, each composed of 316 stainless steel, in series configuration. Each disruption cylinder had a length of 304.8 mm and a nominal internal diameter of 10.9 mm. The eccentric rotating mass weight was 38 g. The disruption motor frequency was 7,300 rpm. The disruption agitation media were solid cylindrical rods made of 316 stainless steel, 300 mm in length and 2 mm in diameter. Each chamber was packed with 21 rods. Fluid was pumped through the system at a rate of 205 mL/min.

For each experiment, samples were taken before and after disruption. Oocysts were enumerated microscopically by size, and the disruption percentage for each size category was calculated. Disruption results for each run in the set of independently prepared and processed suspensions are summarized in Table 29.

TABLE 29
Disruption % at 100 Oocysts/uL
TABLE
Disruption % at 100 Oocysts/uL

| | % Large | % Medium | % Small |
|---|---|---|---|
| | Oocysts | Oocysts | Oocysts |
| Date | Disruption | Disruption | Disruption |
| 2024-08- | 71.74 | 47.39 | 34.63 |
| 15 | | | |
| 2024-09- | 73.40 | 61.36 | 30.12 |
| 05 | | | |
| 2024-09- | 77.26 | 49.02 | 35.96 |
| 26 | | | |
| 2024-10- | 69.19 | 59.05 | 31.49 |
| 15 | | | |
| 2024-10- | 72.54 | 63.27 | 34.57 |
| 31 | | | |
| 2025-01- | 76.64 | 59.39 | 35.24 |
| 07 | | | |
| 2025-02- | 69.85 | 56.88 | 42.13 |
| 13 | | | |
| 2025-04- | 71.28 | 49.17 | 31.21 |
| 03 | | | |
| 2025-04- | 72.33 | 53.96 | 43.19 |
| 24 | | | |
| 2025-05- | 72.39 | 55.49 | 35.89 |
| 13 | | | |
| 2025-05- | 78.03 | 70.37 | 46.81 |
| 20 | | | |
| 2025-05- | 77.99 | 59.74 | 42.36 |
| 27 | | | |
| 2025-06- | 72.86 | 45.01 | 40.41 |
| 03 | | | |
| 2025-06- | 76.41 | 53.88 | 44.47 |
| 10 | | | |
| 2025-06- | 76.87 | 55.46 | 46.11 |
| 17 | | | |
| 2025-06- | 73.94 | 62.75 | 39.76 |
| 24 | | | |
| 2025-07- | 76.35 | 64.50 | 49.91 |
| 08 | | | |
| 2025-07- | 77.85 | 57.04 | 44.48 |
| 15 | | | |
| 2025-07- | 74.68 | 62.39 | 47.22 |
| 22 | | | |

Summary statistics for the set of samples prepared at 100 total sporulated oocysts per microliter are shown in Table 30. Conditions used were tuned primarily to provide efficient disruption of large sporulated oocysts, E. maxima. Results for large oocysts were highly repeatable, with a coefficient of variation of 3.82%. Overall, the vibrating-rod system was demonstrated to be a highly reliable method for disruption of oocysts of varying sizes ranging from approximately 15 μm to 31 μm at either 30 total sporulated oocysts per microliter or 100 total sporulated oocysts per microliter.

TABLE 30
Disruption % Summary Statistics at 100 Oocysts/uL
Disruption % Summary Statistics at 100 oocysts/uL

| | % Large | % Medium | % Small |
|---|---|---|---|
| | Oocysts | Oocysts | Oocysts |
| Statistic | Disruption | Disruption | Disruption |
| # Observations | 19 | 19 | 19 |
| Mean Disruption % | 74.29 | 57.17 | 39.79 |
| Std Dev. | 2.84 | 6.49 | 6.03 |
| CV % | 3.82 | 11.34 | 15.16 |

### Example 4—Disruption of MDCK Cells, First Experiment

Cells were more resistant to disruption on the first try than expected. It had been expected that conditions sufficient to disrupt oocysts would be more than sufficient to disrupt mammalian cells.

The disclosed vibrating-rod disruptor has been used successfully for processing coccidiosis vaccine. This experiment evaluated the disruptor's capability for disrupting MDCK cells, starting with the same parameters used for coccidia disruption, namely 205 mL/min and 7,300 rpm.

The disruption assembly consisted of two hollow cylindrical chambers, each composed of 316 stainless steel, in series configuration. Each disruption cylinder had a length of 304.8 mm and a nominal internal diameter of 10.9 mm. The eccentric rotating mass weight was 38 g. The disruption motor frequency was 7,300 rpm. The disruption agitation media were solid cylindrical rods made of 316 stainless steel, 300 mm in length and 2 mm in diameter. Each chamber was packed with 21 rods. Fluid was pumped through the system at a rate of 205 mL/min.

The disruption system was prepared for operation by flushing the disruptor thoroughly with distilled water, followed by flushing the disruptor with 500 mL of phosphate-buffered saline (PBS). All operational parameters, including flow rate and rod vibration frequency, were recorded. MDCK cells were detached from confluent T75 flasks by trypsinization. The trypsin was neutralized with fetal bovine serum, and the cells were centrifuged at 1500 rpm for 5 minutes. The supernatant was discarded, and the MDCK cells were resuspended in 10 mL of PBS. The MDCK cells were counted using a hemocytometer, with two individuals performing three independent counts. A 1 liter MDCK cell suspension in PBS was generated by dilution of the 10 mL PBS MDCK cell suspension. A 100 mL aliquot, designated the P0 sample, was taken at the beginning and counted using a hemocytometer, with two individuals performing three independent counts. A separate 100 mL aliquot of the 1 L cell suspension was collected and placed on the bench for the duration of the experiment, and that aliquot was counted at the end of the experiment. The remaining 800 mL of the cell suspension was passed through the disruption system as a first pass, designated P1. A 100 mL aliquot was immediately collected from the outflow for analysis.

The P1 sample was counted using a hemocytometer, with two individuals performing three independent counts. Cell concentration was calculated, and the percentage disruption was calculated as the total cells counted in P1 divided by the total number of cells counted in P0. The P1 outflow was then passed through the disruption system as a second pass, designated P2. A 100 mL aliquot was immediately collected from the outflow for analysis. The P2 sample was counted using a hemocytometer, with two individuals performing three independent counts. Cell concentration was calculated, and the percentage disruption was calculated as the total cells counted in P2 divided by the total number of cells counted in P0. The P2 outflow was then passed through the disruption system as a third pass, designated P3. A 100 mL aliquot was immediately collected from the outflow for analysis. The P3 sample was counted using a hemocytometer, with two individuals performing three independent counts. Cell concentration was calculated, and the percentage disruption was calculated as the total cells counted in P3 divided by the total number of cells counted in P0.

Results of the quantification at each step are summarized in Table 31. Overall, the percentage of disruption observed after three passes was 54%.

TABLE 31
Disruption of MDCK cells - First Experiment

| Pass | Volume (mL) | Cell conc. (cells/mL) | Total Cells | % Intact Cells | % Disruption |
|---|---|---|---|---|---|
| P0 (initial) at the end | 100 | 2.39E+05 | 2.39E+07 | 100% | 0% |
| P1 | 100 | 1.60E+05 | 1.60E+07 | 67% | 33% |
| P2 | 100 | 1.43E+05 | 1.43E+07 | 60% | 40% |
| P3 | 100 | 1.09E+05 | 1.09E+07 | 46% | 54% |

### Example 5—Eimeria Disruption Under Harsher Conditions

An experimental system was assembled to produce more complete disruption of Eimeria oocysts. The disruption assembly consisted of two hollow cylindrical chambers, each composed of 316 stainless steel, in series configuration. Each disruption cylinder had a length of 304.8 mm and a nominal internal diameter of 10.9 mm. The eccentric rotating mass weight was 38 g. The disruption motor frequency was 10,250 rpm. The disruption agitation media were solid cylindrical rods made of 304 stainless steel, 300 mm in length and 1 mm in diameter. Each chamber was packed with 85 rods. Fluid was pumped through the system at a rate of 85 mL/min.

Six replicate samples were processed through the system. For each replicate, samples were taken before and after disruption. Oocysts were enumerated microscopically by size, and the disruption percentage for each size category was calculated. Disruption results for each run in the set of independently prepared and processed suspensions are summarized in Table 32.

TABLE 32
Oocyst Counts Data

| | | | Medium | Small |
|---|---|---|---|---|
| Sample | Replicate | Large Oocysts/mL | Oocysts/mL | Oocysts/mL |
| Initial | 1 | 2.68 × 10³ | 9.79 × 10² | 8.75 × 10³ |
| Initial | 2 | 2.83 × 10³ | 9.31 × 10² | 8.96 × 10³ |
| Initial | 3 | 2.48 × 10³ | 1.03 × 10³ | 8.75 × 10³ |
| Initial | 4 | 2.83 × 10³ | 8.78 × 10² | 9.15 × 10³ |
| Initial | 5 | 2.32 × 10³ | 1.23 × 10³ | 8.15 × 10³ |
| Initial | 6 | 2.63 × 10³ | 9.01 × 10² | 8.32 × 10³ |
| Processed | 1 | 8.00 × 10⁰ | 0.00 × 10⁰ | 8.40 × 10¹ |
| Processed | 2 | 0.00 × 10⁰ | 0.00 × 10⁰ | 8.30 × 10¹ |
| Processed | 3 | 0.00 × 10⁰ | 0.00 × 10⁰ | 4.60 × 10¹ |
| Processed | 4 | 0.00 × 10⁰ | 0.00 × 10⁰ | 6.80 × 10¹ |
| Processed | 5 | 0.00 × 10⁰ | 0.00 × 10⁰ | 7.60 × 10¹ |
| Processed | 6 | 0.00 × 10⁰ | 0.00 × 10⁰ | 6.00 × 10¹ |

Summary statistics are presented in Table 33. Results indicate that under the harsher conditions used in this experiment, disruption of oocysts was nearly complete.

TABLE 33
Oocyst Disruption Summary Statistics

| | | | Standard | | | |
|---|---|---|---|---|---|---|
| | | Mean | Deviation | Max | Min | Disruption |
| Sample Type | N | (Oocysts/mL) | (Oocysts/mL) | (Oocysts/mL) | (Oocysts/mL) | (%) |
| Initial Large | 6 | 2.63 × 10³ | 2.02 × 10² | 2.83 × 10³ | 2.32 × 10³ | NA |
| Initial Medium | 6 | 9.92 × 10² | 1.30 × 10² | 1.23 × 10³ | 8.78 × 10² | NA |
| Initial Small | 6 | 8.68 × 10³ | 3.81 × 10² | 9.15 × 10³ | 8.15 × 10³ | NA |
| Processed Large | 6 | 1.33 × 10⁰ | 3.27 × 10⁰ | 8.00 × 10⁰ | 0.00 × 10⁰ | 99.95 |
| Processed Medium | 6 | 0.00 × 10⁰ | 0.00 × 10⁰ | 0.00 × 10⁰ | 0.00 × 10⁰ | 100.00 |
| Processed Small | 6 | 6.95 × 10¹ | 1.47 × 10¹ | 8.40 × 10¹ | 4.60 × 10¹ | 99.20 |

### Example 6—Disruption of MDCK Cells, Second Experiment

The disruptor's capability for MDCK cells was further tested using modified conditions. The following parameters were used: a flow rate of 80 mL/min to 85 mL/min and a vibration frequency of 10,500 rpm, as compared to the standard disruption parameters of 205 mL/min and 7,300 rpm.

The benchtop disruptor was prepared for operation by flushing the disruptor thoroughly with distilled water, followed by flushing the disruptor with 500 mL of phosphate-buffered saline (PBS). All operational parameters, including flow rate and rod vibration frequency, were recorded.

MDCK cells were detached from confluent T75 flasks by trypsinization. The trypsin was neutralized with fetal bovine serum, the cells were centrifuged at 1500 rpm for 5 minutes, the supernatant was discarded, and the MDCK cells were resuspended in 10 mL of PBS. The MDCK cells were counted using a hemocytometer, with two individuals performing three independent counts. A 1 L MDCK cell suspension in PBS was generated by dilution of the 10 mL PBS MDCK cell suspension. A 100 mL aliquot, designated P0, was taken and counted using a hemocytometer, with two individuals performing three independent counts. A separate 100 mL aliquot of the 1 L cell suspension was collected and placed on the bench for the duration of the experiment, and that aliquot was counted at the end of the experiment. The remaining 800 mL of the cell suspension was passed through the disruption system as a first pass, designated P1. A 100 mL aliquot was immediately collected from the outflow for analysis.

The P1 sample was counted using a hemocytometer, with two individuals performing three independent counts. Cell concentration was calculated, and the percentage disruption was calculated as the total cells counted in P1 divided by the total number of cells counted in P0. The P1 outflow was passed through the disruption system as a second pass, designated P2. A 100 mL aliquot was immediately collected from the outflow for analysis. The P2 sample was counted using a hemocytometer, with two individuals performing three independent counts. Cell concentration was calculated, and the percentage disruption was calculated as the total cells counted in P2 divided by the total number of cells counted in P0. The P2 outflow was passed through the disruption system as a third pass, designated P3. A 100 mL aliquot was immediately collected from the outflow for analysis. The P3 sample was counted using a hemocytometer, with two individuals performing three independent counts. Cell concentration was calculated, and the percentage disruption was calculated as the total cells counted in P3 divided by the total number of cells counted in P0. The benchtop disruptor was cleaned after completion of the experiment. Results of the quantification at each step are summarized in Table 34. Using the modified parameters, a high level of cell disruption was observed.

TABLE 34
Disruption of MDCK cells - Modified Conditions

| Pass | Volume (mL) | Cell conc. (cells/mL) | Total Cells | % Intact Cells | % Disruption |
|---|---|---|---|---|---|
| P0 (initial) at the end | 100 | 3.49E+05 | 3.49E+07 | 100 | 0 |
| P1 | 100 | 7.20E+04 | 7.20E+06 | 21 | 79 |
| P2 | 100 | 2.87E+04 | 2.87E+06 | 8 | 92 |
| P3 | 100 | 1.20E+04 | 1.20E+06 | 3 | 97 |

### Non-Limiting Nature of the Disclosure

It should be understood that the above description is representative only of illustrative embodiments and examples. For convenience of the reader, the description above has focused on a limited number of representative examples that illustrate principles of the disclosure. The description has not attempted to exhaustively enumerate all possible variations or combinations of such variations. The absence of discussion of alternate embodiments for a specific portion of the disclosure, or the availability of further undescribed alternate embodiments, is not to be considered a disclaimer of such alternate embodiments. One of ordinary skill in the art will appreciate that many such undescribed embodiments may differ in technology and materials rather than in application of the principles of the disclosure. Accordingly, the disclosure is not intended to be limited except as set forth in the following claims and their equivalents.

### INCORPORATION BY REFERENCE

All references, articles, publications, patents, patent publications, and patent applications cited herein are incorporated by reference in their entireties for all purposes. However, mention of any reference, article, publication, patent, patent publication, or patent application cited herein is not, and should not be taken as, an acknowledgment or any form of suggestion that it constitutes valid prior art or forms part of the common general knowledge in any country in the world. It is to be understood that, while the disclosure has been described in conjunction with the detailed description thereof, the foregoing description is intended to illustrate and not limit the scope. Other aspects, advantages, and modifications are within the scope of the claims set forth below. All publications, patents, and patent applications cited in this specification are incorporated by reference as if each individual publication, patent, or patent application were specifically and individually indicated to be incorporated by reference.

What is claimed is:

1. A method of disrupting outer enclosures of cells and releasing contents therefrom, the method comprising:
providing a system comprising:
a first vessel containing a volume of cells in suspension, the cells having outer enclosures that contain fluid and microstructures of the cells therein;
an inline disruption system comprising a vibration source and a cell processing chamber containing vibrating components configured to disrupt the outer enclosures of at least some of the cells; and
a downstream containment vessel connected to the inline disruption system;
moving the suspension from the first vessel into the inline disruption system;
passing the suspension through the inline disruption system, whereby at least some of the outer enclosures of the cells are disrupted, which results in a release of contents from the cells, thereby producing a modified solution comprising the released contents; and
the modified solution from the inline disruption system to the downstream containment vessel or to other system components for additional processing, wherein the modified solution is one of: held in the downstream containment vessel, passed again through the inline disruption system one or more times, delivered to a delivery device for delivery to a subject, or directed to other system components for additional processing.

2. The method of claim 1, wherein the cell processing chamber contains vibrating components comprising at least one selected from the group consisting of vibrating plates in a rectangular chamber, a single vibrating rod in a cylindrical chamber, a single vibrating rod split lengthwise in a cylindrical chamber, a plurality of vibrating rods in a cylindrical chamber, concentric cylinders with a central rod in a cylindrical chamber, and concentric cylinders with a central rod split lengthwise in a cylindrical chamber.

3. The method of claim 2, wherein the cells are Apicomplexan parasites.

4. The method of claim 3, wherein the Apicomplexan parasites are Eimeria oocysts, wherein the released contents include viable sporocysts, and wherein the modified solution is moved to a delivery system comprising a nozzle or an orifice plate for delivery to individual chickens in a high-throughput, in-motion vaccination system.

5. The method of claim 4, wherein the modified solution is delivered to facial mucosa of the individual chickens within a 24-hour period of time after the viable sporocysts are released.

6. The method of claim 2, wherein the cells are selected from the group consisting of mammalian cells, avian cells, bacterial cells, yeast cells, fungal cells, insect cells, plant cells, tissues, and organoids.

7. The method of claim 2, wherein the cells originate from a complex source comprising at least one selected from the group consisting of unprocessed algal biomass, processed algal biomass, municipal waste, and poultry litter.

8. The method of claim 7, wherein the complex source comprises biomass containing recoverable biofuel precursors.

9. The method of claim 7, wherein the complex source comprises material containing recoverable phosphorus-containing and/or nitrogen-containing compounds.

10. The method of claim 7, wherein the cells are from a source material processed for use in human food or animal feed.

11. The method of claim 7, wherein the plant cells or fungal cells contain accumulated mineral material for phytomining or mycomining.

12. A system for disrupting outer enclosures of cells and releasing contents therefrom, the system comprising:
a first vessel containing cells in suspension, the cells having outer enclosures that contain fluid and microstructures of the cells therein;
an inline disruption system comprising a vibration source and a cell processing chamber containing vibrating components, the cell processing chamber being configured to receive the suspension from the first vessel and to modify the suspension by disrupting the outer enclosures of at least some of the cells, thereby releasing contents from the disrupted cells and producing a modified solution; and
an outlet connected to the cell processing chamber and configured to deliver the modified solution to a downstream containment vessel or to other system components for additional processing.

13. The system of claim 12, wherein the cell processing chamber contains vibrating components comprising at least one selected from the group consisting of vibrating plates in a rectangular chamber, a single vibrating rod in a cylindrical chamber, a single vibrating rod split lengthwise in a cylindrical chamber, a plurality of vibrating rods in a cylindrical chamber, concentric cylinders with a central rod in a cylindrical chamber, and concentric cylinders with a central rod split lengthwise in a cylindrical chamber.

14. The system of claim 13, configured to disrupt Apicomplexan parasites.

15. The system of claim 14, wherein the Apicomplexan parasites are Eimeria oocysts, wherein the released contents include viable sporocysts, and wherein the system is configured to deliver the modified solution to a delivery system comprising a nozzle or an orifice plate for delivery to individual chickens in a high-throughput, in-motion vaccination system.

16. The system of claim 14, wherein the Apicomplexan parasites are Plasmodium oocysts and the disruption releases viable Plasmodium sporozoites.

17. The system of claim 15, wherein the delivery system is configured to deliver the modified solution to facial mucosa of the individual chickens within a 24-hour period of time after the viable sporocysts are released.

18. The system of claim 15, wherein surfaces of the cell processing chamber, the vibrating rods, and the nozzle or orifice plate are independently prepared as either untreated, wear resistant, hydrophilic, or hydrophobic.

19. The system of claim 14, configured to disrupt cells selected from the group consisting of mammalian cells, avian cells, bacterial cells, yeast cells, fungal cells, insect cells, plant cells, tissues, and organoids.

20. The system of claim 14, configured to disrupt cells originating from a complex source comprising at least one selected from the group consisting of unprocessed algal biomass, processed algal biomass, municipal waste, and poultry litter.

21. The system of claim 20, wherein the complex source comprises biomass containing recoverable biofuel precursors.

22. The system of claim 20, wherein the complex source comprises material containing recoverable phosphorus-containing and/or nitrogen-containing compounds.

23. The system of claim 20, wherein the cells are from a source material processed for use in human food or animal feed.

24. The system of claim 20, wherein the plant cells or fungal cells contain accumulated mineral material for phytomining or mycomining.

25. A system for disrupting outer membranes of Plasmodium oocysts and releasing viable sporozoites therefrom, the system comprising:
a vessel containing Plasmodium oocysts in a solution, the Plasmodium oocysts having outer membranes and containing viable sporozoites therein;
an inline disruption system comprising a vibration source and an oocyst processing chamber containing vibrating rods, the oocyst processing chamber being configured to receive the solution from the vessel and to modify the solution by disrupting the outer membranes of at least some of the Plasmodium oocysts, thereby releasing viable sporozoites from the disrupted Plasmodium oocysts and producing a modified solution; and
an outlet connected to the oocyst processing chamber and configured to deliver the modified solution downstream for further processing.
